(19) United States
(12) Patent Application Publication
(10) Pub. No.: US 2006/0222672 A1
(43) Pub. Date: Oct. 5, 2006

(54) Gaseous constituent supply device for vehicle

(75) Inventors: Tomohisa Ezaka, Kariya-city (JP); Shigeki Harada, Toyota-city (JP); Yukiya Sassa, Nagoya-city (JP); Manabu Maeda, Nagoya-city (JP); Takurou Kawaguchi, Kariya-city (JP)

Correspondence Address: HARNESS, DICKEY & PIERCE, P.L.C., P.O. BOX 828, BLOOMFIELD HILLS, MI, 48303, US

(73) Assignee: DENSO Corporation, Kariya-city (JP)

(21) Appl. No.: 11/393,036

(22) Filed: Mar. 30, 2006

(30) Foreign Application Priority Data
Apr. 1, 2005 (JP) 2005-106852
Apr. 1, 2005 (JP) 2005-106849
Jan. 24, 2006 (JP) 2006-015625

(51) Int. Cl. A61L 9/00; A61M 11/00

(52) U.S. Cl. 424/401; 424/76.2; 128/200.23

(57) ABSTRACT

A gaseous constituent supply device supplies an air cannon projectile containing a predetermined gaseous constituent to an occupant in a compartment of a vehicle. This supply device includes a gaseous constituent supply chamber for reserving the gaseous constituent, an air compression unit for compressing an inside of the gaseous constituent supply chamber to generate an air cannon projectile including the gaseous constituent and to emit the air cannon projectile to an individual occupant, and a control unit that estimates an airflow state in the compartment and controls operation of the air compression means based on the estimated airflow state.

### CROSS REFERENCE TO RELATED APPLICATION

This application is based on Japanese Patent Applications No. 2005-106852 filed on Apr. 1, 2005, No. 2005-106849 filed on Apr. 1, 2005, and No. 2006-15625 filed on Jan. 24, 2006, the contents of which are incorporated herein by reference in its entirety.

### FIELD OF THE INVENTION

This invention relates to a gaseous constituent supply device for a vehicle, for supplying an air cannon projectile (e.g., air vortex ring) containing a predetermined gaseous constituent to an occupant in a compartment of the vehicle.

### BACKGROUND OF THE INVENTION

Conventionally, there is known a perfume apparatus that blends a perfume constituent into air-conditioning air to be blown from an air conditioner outlet port and blows it into the compartment of a vehicle whereby an unpleasant odor in the vehicle is eliminated and comfortable feeling of a scent is provided to the occupant of the compartment of the vehicle.

This device is configured to blow air-conditioning air containing a perfume constituent from the outlet port provided in an instrument panel so as to supply the perfume constituent to the whole compartment. With such a configuration, since it is not necessary to provide separately a mechanism for blowing of air containing a perfume constituent, a perfume is prevailed all over the whole compartment (for example, JP Patent No. 3134306).

Since a time when each occupant wants to have a perfume constituent and an amount of the perfume constituent that is sensed comfortable to each occupant are different among the occupants, a structure capable of providing a perfume constituent independently for the occupant is being sought. Since the conventional perfume apparatus described above is configured to blow air-conditioning air containing a perfume constituent from the air conditioner outlet port, there was a problem that each of the occupants is not provided with an optimum perfume constituent.

In a conceivable case where the apparatus is configured so as to emit air containing the perfume constituent to each of the occupants, the perfume constituent may be diffused by the air-conditioning air blown from the air conditioner outlet port or by the external airflow that invades through side window glass. Since it is considered that this phenomenon makes it difficult to provide a perfume constituent to the occupant, countermeasures against them need to be examined.

### SUMMARY OF THE INVENTION

This invention is made in view of the above-mentioned problem, and has its object to provide a gaseous constituent supply device for a vehicle capable of supplying a gaseous constituent to each individual occupant.

According to an aspect of the present invention, a gaseous constituent supply device for supplying an air cannon projectile containing a predetermined gaseous constituent to an occupant in a compartment of a vehicle includes a gaseous constituent supply chamber for reserving the gaseous constituent, an air compression means for compressing an inside of the gaseous constituent supply chamber to generate an air cannon projectile including the gaseous constituent and to emit the air cannon projectile to an individual occupant, and a control unit that estimates an airflow state in the compartment and controls operation of the air compression means based on the estimated airflow state.

Accordingly, the gaseous constituent supply device can emit the air cannon projectile containing a predetermined gaseous constituent toward each of the occupants, and consequently an optimum gaseous constituent can be supplied to each of the occupants. Moreover, since the gaseous constituent supply device is configured to control operation of the air compressing means, this gaseous constituent can be supplied to any one of the occupants who wants to have this gaseous constituent. Furthermore, it can prevent an air cannon projectile from being diverted from a direction of the occupant by a generated airflow resulting from an operating state of an in-vehicle apparatuses; and prevent the air cannon projectile from diffusing before reaching the occupants.

For example, the control unit permits operation of the air compression means when a set air volume of air-conditioning air being blown from an outlet port of an air conditioner of the vehicle is less than a predetermined amount. In contrast, the control unit stops the operation of the air compression means when the set air volume is larger than the predetermined amount.

Alternatively, the control unit permits operation of the air compression means when a blowing direction of air-conditioning air being blown from an outlet port of an air conditioner is set to be in a direction diverted from the occupant; and the control unit stops the operation of the air compression means when the blowing direction is set to be in a direction toward the occupant. Alternatively, the control unit permits operation of the air compression means when a window glass of the vehicle is set in a close state, and the control unit stops the operation of the air compression means when the window glass is set in an open state. Alternatively, the control unit permits operation of the air compression means when a sunroof of the vehicle is set in a close state, and the control unit stops the operation of the air compression means when the sunroof of the vehicle is set in an open state.

The gaseous constituent supply device can be provided with an airflow sensor for detecting an airflow. In this case, the control unit permits operation of the air compression means when the control unit determines a non-airflow state in the compartment based on the detected value of the airflow sensor, and the control unit stops the operation of the air compression means when the control unit estimates an airflow state in the compartment based on the detected value of the airflow sensor. The airflow sensor can be disposed to detect a flow velocity of the airflow. In this case, the control unit permits the operation of the air compression means when the control unit estimates that the flow velocity of the airflow is not more than a predetermined velocity; and the control unit stops the operation of the air compression means when the control unit estimates that the flow velocity is more than the predetermined velocity.

A state detecting sensor for detecting a seating state of the occupant can be provided. In this case, the control unit controls operation of the air compression means depending on a change in the seating state. Further, the control unit can estimate an airflow state based on an operating state of an in-vehicle apparatus in a timing in which the air cannon projectile is emitted. In this case, the control unit controls an operation of the air compression means such that the air cannon projectile reaches the occupant even in an airflow generation state in which an airflow is generated in the compartment when the control unit estimates the airflow generation state. For example, the in-vehicle apparatus is an air conditioner for air-conditioning the compartment by blowing air-conditioning air to the compartment, and the control unit estimates the airflow state based on at least one of a blowing direction and an air volume of the air-conditioning air. Alternatively, the in-vehicle apparatus is a window glass of the vehicle, and the control unit estimates the airflow state based on an open degree of the window glass. Alternatively, the in-vehicle apparatus is a sunroof of the vehicle, and the control unit estimates the airflow state based on the open degree of the sunroof.

Furthermore, the control unit can estimate an airflow state in the compartment based on an operating state of an in-vehicle apparatus in a timing of emitting the air cannon projectile. In this case, when it is estimated to be an airflow generation state in which an airflow is generated in the compartment, the control unit reduces the airflow generation state. In contrast, the control unit controls an operating state of the in-vehicle apparatus such that the air cannon projectile reaches the occupant even in the airflow generation state.

The control unit can presume the airflow state in the compartment based on at least one of a blowing direction and an air volume of the air-conditioning air. In this case, when the control unit estimates that the airflow state is an airflow generation state, the control unit controls at least one of the blowing direction and the air volume of the air-conditioning air. Further, the control unit can control the air conditioner such that the blowing direction of the air-conditioning air approaches the same direction as an emission direction of the air cannon projectile and a flow velocity of the air-conditioning air approaches the same as a flow velocity of the air cannon projectile.

The gaseous constituents can include a plurality of perfume constituents, and perfume constituent supply means can supply selectively the plurality of perfume constituents as the air cannon projectile to the gaseous constituent supply chamber. Furthermore, the control unit can determine a perfume constituent selected from among the plurality of perfume constituents based on a signal of a navigation ECU of the vehicle or a signal of a vehicular integrated ECU.

The control unit can determine a perfume constituent selected from among the plurality of perfume constituents based on detection data of a temperature sensor for detecting a body temperature of the occupant. Alternatively, the control unit determines a perfume constituent selected from among the plurality of perfume constituents based on detection data of a biomedical sensor for detecting a biomedical signal of the occupant.

Furthermore, the gaseous constituent supply device can be provided with an operation selection switch capable of selecting an operation or operation stop of the air compression means. In this case, the control unit operates based on an item being set up by the operation selection switch. Alternatively, the gaseous constituent supply device can be provided with a perfume constituent selection switch capable of selecting at least one perfume constituent from among the plurality of perfume constituents. In this case, the perfume constituent supply means operates based on an item being set up by the perfume constituent selection switch.

The gaseous constituent supply chamber and the air compression means can be constructed in an enclosure. In this case, the air compression means can include a compression member for changing a volume of the gaseous constituent supply chamber by magnetic force and electromagnetic force, a magnet provided in the compression member, and a coil installed facing the compression member.

Further, a humidity constituent generating unit for generating a humidity constituent and a humidity sensor for detecting a humidity of air in the compartment can be provided. In this case, the control unit controls the humidity constituent generating unit to supply the air cannon projectile including the humidity constituent into the compartment when a detection value of the humidity sensor is lower than a predetermined value.

According to another aspect of the present invention, a gaseous constituent supply device for supplying an air cannon projectile containing a predetermined gaseous constituent to an occupant in a compartment of a vehicle, includes a humidity constituent generating unit for generating a humidity constituent to be contained in the air cannon projectile, a gaseous constituent chamber for reserving the gaseous constituent, air compression means for compressing an inside of the gaseous constituent chamber to generate an air cannon projectile including the humidity constituent, a humidity sensor for detecting a humidity of air in the compartment, and a control unit which controls the humidity constituent generating unit and the compression means so that an air cannon projectile including a predetermined amount of the humidity constituent is emitted to the occupant when the humidity of air detected by the humidity sensor is lower than a predetermined value. Accordingly, pleasant feeling can be given to the occupant in the compartment of the vehicle. In this case, the humidity constituent generating unit includes a water supply unit for supplying water and a heating unit for evaporating the water supplied from the water supply unit, and the humidity constituent generating unit can be disposed in the gaseous constituent chamber.

According to another aspect of the present invention, a gaseous constituent supply device for supplying an air cannon projectile containing a predetermined gaseous constituent to an occupant in a compartment of a vehicle, includes an enclosure having therein a gaseous constituent chamber for reserving the gaseous constituent and air compression means for compressing an inside of the gaseous constituent chamber to generate an air cannon projectile including the predetermined gaseous constituent. Furthermore, the air compression means includes a compression member for changing a volume of the gaseous constituent chamber by magnetic force and electromagnetic force, a magnet provided in the compression member, and a coil installed facing the compression member. For example, the compression member is a plate member having an elasticity, and the electromagnetic force of the compression means is generated when a square-wave voltage is applied to the compression means. Furthermore, the compression means can have a booster circuit through which the square-wave voltage is applied.

### BRIEF DESCRIPTION OF THE DRAWINGS

Additional objects and advantages of the present invention will be more readily apparent from the following detailed description of preferred embodiments when taken together with the accompanying drawings.

FIG. 1 is a schematic diagram showing an in-vehicle arrangement structure of a gaseous constituent emitting unit in a first embodiment.

FIG. 2 is a block diagram showing the whole configuration of a gaseous constituent supply device for a vehicle.

FIG. 3 is a schematic diagram showing an internal structure of a gaseous constituent supply chamber and an air compression means of an example of the first embodiment.

FIG. 4 is a perspective view showing the internal structure of the gaseous constituent supply chamber and the air compression means with some parts of them broken and taken away.

FIG. 5 is a schematic diagram showing an internal structure of a gaseous constituent supply chamber and an air compression means of another example of the first embodiment.

FIG. 6 is a perspective view showing the internal structure of a gaseous constituent supply chamber and an air compression means with some parts of them broken and taken away in the another example.

FIG. 7 is a schematic diagram showing in-vehicle arrangement structure of an infrared sensor.

FIG. 8 is a flowchart showing a control of a gaseous constituent supply ECU.

FIG. 9 is a schematic diagram showing in-vehicle arrangement structure of an airflow sensor that partly constitutes a gaseous constituent supply device for a vehicle of a second embodiment.

FIG. 10 is a block diagram showing the whole configuration of the gaseous constituent supply device for a vehicle of the second embodiment.

FIG. 11 is a flowchart showing a control of a gaseous constituent supply ECU in a third embodiment.

FIG. 12 is a flowchart showing a control of a gaseous constituent supply ECU in a fourth embodiment.

FIG. 13 is a flowchart showing a control of a gaseous constituent supply ECU in a fifth embodiment.

FIG. 14 is a block diagram showing the whole configuration of a gaseous constituent supply device for a vehicle in a sixth embodiment.

FIG. 15 is a flowchart showing a control of the gaseous constituent supply ECU of the sixth embodiment.

FIG. 16 is a block diagram showing the whole configuration of a gaseous constituent supply device for a vehicle in a seventh embodiment.

FIG. 17 is a flowchart showing a control of the gaseous constituent supply ECU of the seventh embodiment.

FIG. 18 is a flowchart showing a control of a gaseous constituent supply ECU in an eighth embodiment.

FIG. 19 is a block diagram showing the whole configuration of a gaseous constituent supply device for a vehicle in a ninth embodiment.

FIG. 20 is a schematic diagram showing a disposing location of a humidity sensor of the ninth embodiment.

FIG. 21 is a flowchart showing a control of a gaseous constituent supply ECU of the ninth embodiment.

FIG. 22 is a schematic diagram showing a configuration of a modification of a humidity constituent generating unit.

### DETAILED DESCRIPTION OF THE PREFERRED EMBODIMENTS

### First Embodiment

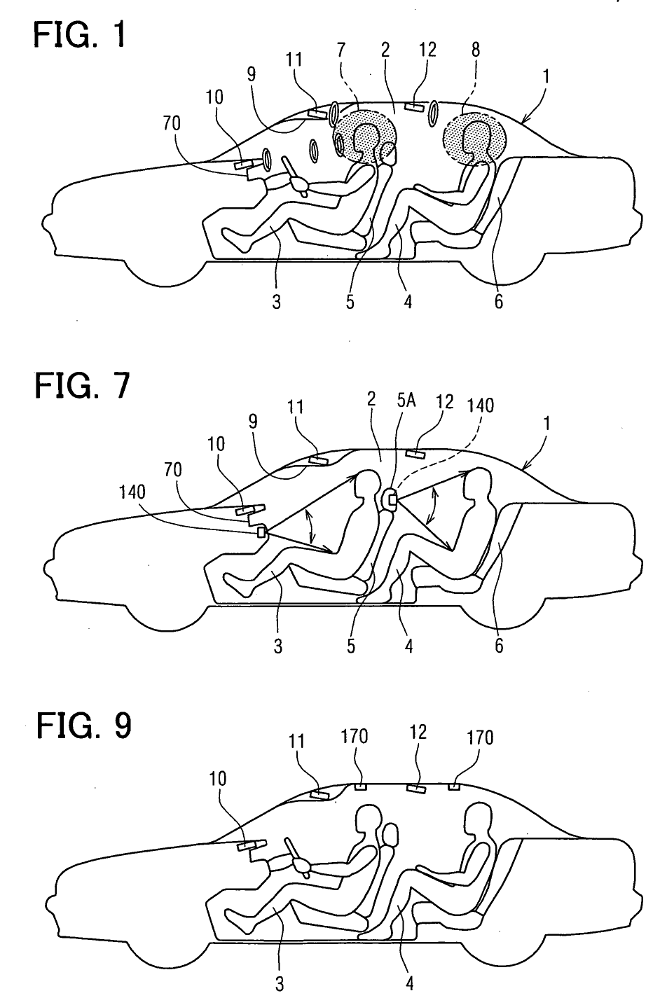

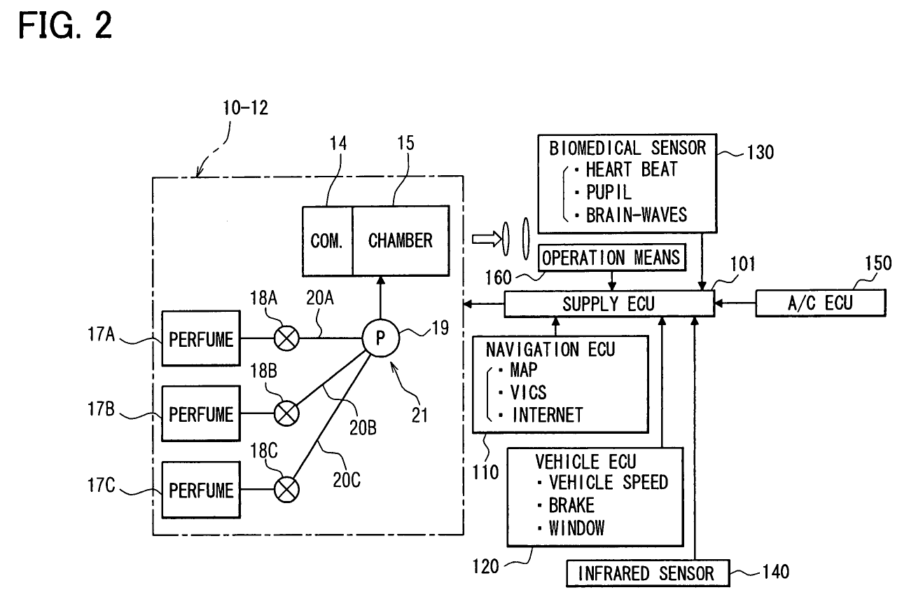

The first embodiment, in which a gaseous constituent supply device for a vehicle according to this invention is typically applied to an automobile, will be described with reference to FIGS. 1 to 8. A gaseous constituent supply device for a vehicle of this embodiment emits an air cannon projectile containing a predetermined gaseous constituent in an occupant directions, and supplies it to occupants 3, 4. The gaseous constituent supply device is constructed with gaseous constituent emitting units 10, 11,12 each for emitting an air cannon projectile unerringly to the occupants 3, 4 who exist in a compartment 2 of an automobile 1, and a gaseous constituent supply ECU 101 (control means) for controlling operations of these units 10, 11,12 (see FIG. 2).

As shown in FIG. 1, the gaseous constituent emitting units 10-12 are installed in positions in which air cannon projectiles can be emitted unerringly to the occupants 3, 4 who exist in the compartment 2.

An air cannon projectile in this invention means one such that a fluid reserved in a certain space is compressed, pushed out from an emission port formed in that space, and emitted out therefrom becoming a mass of the fluid. Forms of the air cannon projectiles include all of what are emitted in a mass, for example, in the form of a vortex ring, a sphere, etc.

The gaseous constituent emitting unit 10 is installed in an instrument panel 70 to be able to emit an air cannon projectile toward the compartment 2. Note that the gaseous constituent emitting unit 10 may be installed in the proximity to the air conditioner normally set up in the instrument panel 70, or may be constructed to be integral with this air conditioner.

An air cannon projectile emitted from an emission port 27 of the gaseous constituent emitting unit 10 is emitted toward the face and its vicinity of the occupant 3 sitting in a front seat 5 via the grilled outlet port formed in the instrument panel 70. Then, when this air cannon projectile hits the face, the shoulder, etc. of the occupant 3, the mass of the air cannon projectile will collapse and the gaseous constituent contained therein will diffuse in a diffusion area 7.

In addition, the gaseous constituent emitting unit 11 is installed in an overhead module 9 formed at the front seat side ceiling part in the compartment 2. The air cannon projectile emitted from emission port of this gaseous constituent emitting unit 11 is emitted toward the face and its vicinity of the occupant 3 sitting in the front seat 5 via the outlet port formed in the overhead module 9. Then, when this air cannon projectile hits the face, the head, etc. of the occupant 3, the mass of the air cannon projectile will collapse and the gaseous constituent contained therein will diffuse in the diffusion area 7.

In addition, the gaseous constituent emitting unit 12 is installed in a rear-seat-side ceiling part in the compartment 2. The air cannon projectile emitted from the emission port of this gaseous constituent emitting unit 12 is emitted toward the face and its vicinity of the occupant 4 sitting in a rear seat 6. Then, when this air cannon projectile hits the face, the head, etc. of the occupant 4, the mass of the air cannon projectile will collapse and the gaseous constituent contained therein will diffuse in a diffusion area 8.

The enclosure that is constructed with the gaseous constituent emitting units 10, 11, 12 as internal constituents is formed in a flat shape, which enables it to be disposed in a narrow space, such as inside the instrument panel 70, inside the overhead module 9, or inside the ceiling part of the compartment 2.

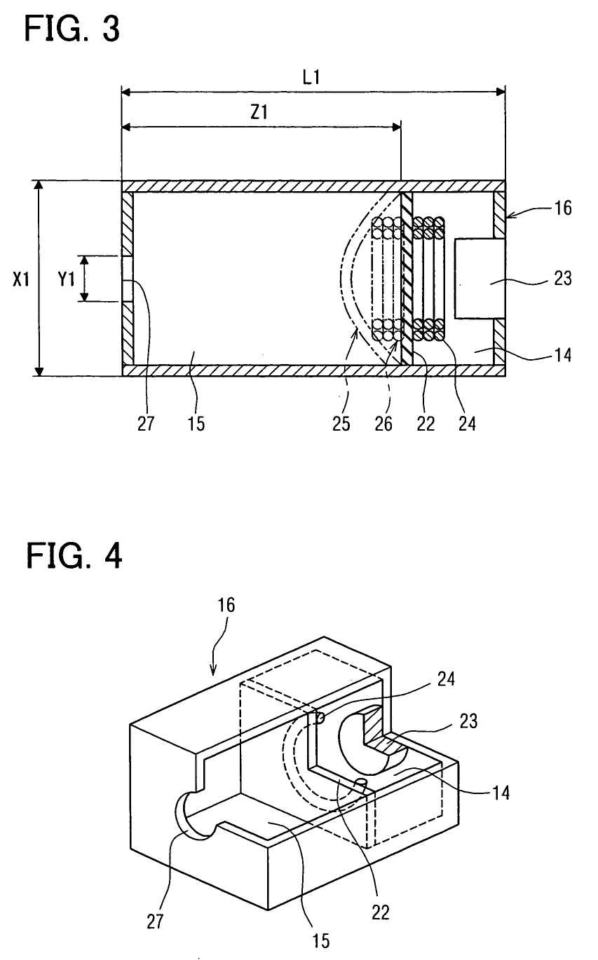

Next, a structure of the gaseous constituent emitting units 10-12 will be explained. As shown in FIG. 3, each of the gaseous constituent emitting units 10-12 has the emission port 27 of the air cannon projectile on an end face on one side of an enclosure 16. Each of the gaseous constituent emitting units 10-12 is constructed of a gaseous constituent supply chamber 15 that communicates with the emission port 27, air compression means 14 for compressing a volume of the gaseous constituent supply chamber 15 on the other side of the gaseous constituent supply chamber 15, perfume constituent reserving means 17A-17C for reserving two or more kinds of perfume constituents, and conveyance means 21 for conveying perfume constituents reserved in these perfume constituent reserving means 17A-17C selectively to the gaseous constituent supply chamber 15 in the enclosure.

As shown in FIG. 3, the gaseous constituent supply chamber 15 is a space to which the perfume constituents reserved in the perfume constituent reserving means 17A-17C are fed by the conveyance means 21 and where it is reserved. Any perfume constituent conveyed by the conveyance means 21 is fed into the gaseous constituent supply chamber 15 as it is in a gaseous state and made to fill this volume.

On the other hand, the air compression means 14 is constructed of a compression member 22 that deforms to the gaseous constituent supply chamber 15 side by magnetic force and electromagnetic force to change the volume of the gaseous constituent supply chamber 15, a coil 24 provided in this compression member 22, and a magnet 23 provided facing the compression member 22.

When voltage is applied to the air compression means 14 from an in-vehicle battery or the like, a current will flow in the coil 24 to exert electromagnetic force that pushes out the compression member 22 to the gaseous constituent supply chamber 15 side. Furthermore, a magnetic force passing through a tubular inside of the coil 24 will also work by the magnet 23, which causes the compression member 22 to deform largely to the gaseous constituent supply chamber 15 side instantaneously. As a result, the compression member 22 will project to the gaseous constituent supply chamber 15 side, up to a position 25, with a central part of the compression means 22 being protruded largely, and the coil 24 will also project to the gaseous constituent supply chamber 15 side, up to a position 26, together with the compression member 22. Since this deformation of the compression member 22 causes the volume of the gaseous constituent supply chamber 15 to become small instantaneously, the perfume constituent filling the inside of the volume will be compressed and emitted to the outside instantaneously from the emission port 27. By making this series of operations occur in a very short time, air containing a perfume constituent emitted from the emission port 27 is emitted into the compartment 2 in a mass of a fluid, for example, in the form of a vortex ring, a sphere, etc.

FIG. 4 is a perspective view showing the inner structure of the enclosure 16, in which the gaseous constituent supply chamber 15 and the air compression means 14 are partitioned by the thin-plate compression member 22. The compression member 22 shall be made up of a tabular member with self-restoration force, and is preferably made up of a thin plate, such as of a rubber, silicone, and an elastomer. The coil 24 may have a structure of being fixed to this compression member 22 with an adhesive or embedded in the compression member 22 to be integral therewith. In a state in which the compression member 22 is not deformed, the magnet 23 is disposed to maintain such a distance to the coil 24 that restricts the magnet 23 from coming in contact with the coil 24. Preferably, the air compression means 14 is so designed that the distance from the surface of the compression member 22 to the magnet 23 is made as small as possible.

Each of the perfume constituent reserving means 17A-17C is a tank for reserving and sealing a perfume constituent that is a gaseous constituent, reserving a perfume constituent having volatility in the ambient atmospheric pressure in a liquid state. Perfume constituents reserved in the respective reserving means 17A-17C differ from one another. For example, the perfume constituent reserving means 17A reserves a constituent of an arousing effect (i.e., a constituent of a strong perfume, a perfume constituent with high-concentration oxygen, a perfume constituent with cold air), 17B reserves a constituent of a relaxing effect (i.e., an aromatic constituent and a perfume constituent with minus ions), and 17C reserves a constituent of a counter carsickness effect, respectively. Moreover, the perfume constituent reserving means 17A-17C are equipped with the respective valves 18A-18C that will be described later, each of which can allow the perfume constituent to emit by a releasing operation of corresponding valves 18A-18C. Note that, although the three perfume constituent reserving means 17A-17C are provided in this embodiment, and are designed to reserve three kinds of perfume constituents. However, the perfume constituent reserving means may be designed to be able to reserve two or less kinds of perfume constituents, or four or more kinds of perfume constituents.

The conveyance means 21 is constructed with the valves 18A-18C, connection tubes 20A-20C for connecting the valves 18A-18C and the gaseous constituent supply chamber 15, and a conveyance pump 19 for conveying perfume constituents to the gaseous constituent supply chamber 15. The valves 18A-18C perform open/close operations in response to driving signals from the gaseous constituent supply ECU 101 that will be explained later. When this is set to be in the open state, a channel to the gaseous constituent supply chamber 15 is opened, and accordingly perfume constituents of the reserving means 17A-17C evaporate and flow into the connection tubes 20A-20C. The conveyance pump 19 operates similarly in response to a driving signal from the gaseous constituent supply ECU 101. By this operating, perfume constituents staying in the connection tubes 18A-18C are fed into the gaseous constituent supply chamber 15. Here, the above-mentioned perfume constituent reserving means 17A-17C and the conveyance means 21 constitute the perfume constituent supply means.

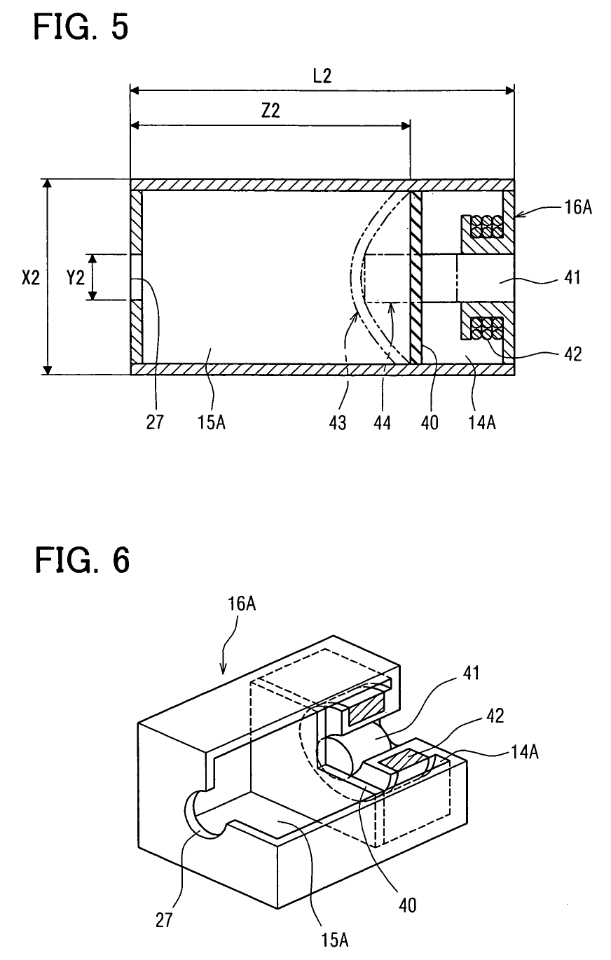

Next, another examples will be described regarding the structure of the gaseous constituent supply means 10-12. As shown in FIG. 5, in the gaseous constituent emitting unit 10-12, a structure of air compression means 14A inside an enclosure 16A is different from the above-mentioned structure. Different parts thereof will be explained below.

This air compression means 14A is constructed of a compression member 40 that deforms to the gaseous constituent supply chamber 15A side by magnetic force and electromagnetic force so as to change a volume of the gaseous constituent supply chamber 15A, a magnet 41 installed in this compression member 40, and a coil 42 installed facing the compression member 40.

When a voltage is applied to the air compression means 14A from an in-vehicle battery or the like, a current flows in the coil 42 to exert an electromagnetic force that pushes the compression member 40 to the gaseous constituent supply chamber 15A side. Moreover, the magnet 41 exerts a repulsion force. With operations of the electromagnetic force and the magnetic force, a force that deforms the compression member 40 will exert accumulatively, and accordingly the compression member 40 will deform largely to the gaseous constituent supply chamber 15A side instantaneously. At this time, the compression member 40 will project to the gaseous constituent supply chamber 15A side, up to a position 43, with a central part of the compression means 40 being protruded largely, and the magnet 41 will also project to the gaseous constituent supply chamber 15A side, up to a position 44, together with the compression member 40. Since this deformation of the compression member 40 makes the volume of the gaseous constituent supply chamber 15A small instantaneously, the perfume constituent filling this volume will be compressed and emitted to the outside from the emission port 27 instantaneously. By making this series of operations take place in a very short time, air containing a perfume constituent emitted from the emission port 27 is emitted into the compartment 2, in a mass of a fluid, for example, in the form of a vortex ring, a sphere, etc.

FIG. 6 is a perspective view showing an internal structure of the enclosure 16A, which is partitioned into the gaseous constituent supply chamber 15A and the air compression means 14A by the thin-plate compression member 40. The coil 42 is disposed to surround the periphery of the magnet 41. The magnet 41 and the coil 42 are insulated by a resin member formed in the shape of a tube. The cylindrical magnet 41 is disposed along the inner diameter of this resin member and the magnet 41 slides along the inner diameter part of the resin member together with deformation of the compression member 40 caused by the electromagnetic force and the magnetic force.

The compression member 40 is made up of a tabular member with a self-restoration force. It is preferable that the compression member 40 is made up of a thin plate such as of a rubber, silicone, and an elastomer. The magnet 41 may be designed to be fixed to this compression member 40 with an adhesive or is embedded in the compression member 40 to be integral therewith. The coil 42 is disposed to maintain such a distance to the compression member 40 that keeps it not to contact with the coil 42. Preferably the air compression means 14A is so configured that the distance between the surface of the compression member 40 and an end face of the enclosure 16A is as small as possible but at the same time the magnet 41 is allowed to be housed in-between.

In the structure of the air compression means 14A, since the magnet 41 is able to travel together with the compression member 40, a movable structured member that excels in strong endurance against repeated reciprocal movement can be obtained.

By constructing the gaseous constituent emitting unit in this way, constitutions of the enclosures 16 and 16A can be formed with a very small size. For example, it is possible to form members of the air compression means 14, 14A plus the gaseous constituent supply chambers 15, 15A, respectively, with the following dimensions: total lengths in the longitudinal direction L1 (L2) are about 50 mm; lengths of the gaseous constituent supply chambers in the longitudinal direction Z1 (Z2) are about 30 mm; widthwise lengths of the enclosures X1 (X2) are about 30 mm; and diameters Y1 (Y2) of the emission port 27 are about φ8-10 mm. Materials of the enclosures 16 and 16A are not limited particularly, but they shall be of materials that can maintain air tightness for some degree, such as a resin and a metal, so that a perfume constituent that fills the gaseous constituent supply chamber 15 may not leak.

A voltage applied to the above-mentioned air compression means 14 (14A) shall be a square-wave voltage. The reason is that this voltage generates an electromagnetic force in a short time and causes the compression member 22 to be deformed instantaneously because the voltage is rich in high-frequency components as compared to a sine wave voltage, which enables a current to flow in the coil 24 instantaneously. Note here that, a square-wave voltage to be applied shall be one that has a pulse width of about 1-sec and a pulse interval of a few sec. Since adoption of a method for applying such a voltage enables a plurality of air cannon projectiles to be emitted toward the occupants 3, 4 continuously, even when a certain obstacle exists on the way, at least any one of the air cannon projectiles hits the occupant 3,4, and consequently the probability that the occupants 3, 4 are made to sense the perfume constituent can be increased.

A voltage to be applied may be a 12-V voltage by supplying it from, for example, an in-vehicle battery. Moreover, in order to boost a voltage to be applied to the air compression means 14, a booster circuit may be used. This booster circuit boosting the applied voltage to, for example, 16 V, can make a conveyance distance of an air cannon projectile larger. Therefore, due to the booster circuit, a gaseous constituent supply device with an enlarged width of selection of the occupants 3, 4 to which the operator wishes to supply an air cannon projectile can be provided.

The gaseous constituent emitting units 10-12 arranged in this way in the compartment 2 are controlled by the gaseous constituent supply ECU 101 that operates based on signals from various apparatuses. The gaseous constituent supply ECU 101 (supply ECU) performs various analysis on signals from a navigation ECU 110, a vehicular integrated ECU 120 (vehicle ECU), a biomedical sensor 130, an infrared sensor 140 (state detection sensor), and an air conditioner ECU 150 (A/C ECU) and/or operation means 160, and determines the following emission conditions: a timing in which an air cannon projectile is emitted; the kind and the amount of a perfume constituent to be contained in the air cannon projectile; an emission velocity of the air cannon projectile; and the like (see FIG. 2).

The gaseous constituent supply ECU 101 performs a control based on the emission conditions to the gaseous constituent emitting units 10-12. This control includes, for example, a control of the amounts of perfume constituents reserved in the perfume constituent reserving means 17A-17C, an adjustment control of the open degree of the valves 18A-18C that constitute the conveyance means 21 for feeding a perfume constituent to the gaseous constituent supply chamber 15, a control of the conveyance pump 19, and a control of the air compression means 14.

Moreover, the gaseous constituent supply ECU 101 controls the air compression means 14 of the gaseous constituent emitting units 10-12 regarding magnitude of the compression force to compress air inside the gaseous constituent supply chamber 15 and starting of the compression force. For example, by controlling the magnitude of the compression force, an air cannon projectile can be controlled as to which occupant of the occupants 3, 4 will receive the emission of an air cannon projectile and how far the air cannon projectile is made to reach. By controlling the starting of the compression force, the air cannon projectile can be controlled as to whether the air cannon projectiles are emitted continuously and whether the air cannon projectile are emitted intermittently with intervals of a predetermined time being set between emissions. Moreover, as shown in FIG. 1, when two or more gaseous constituent emitting units 10-12 are arranged in the compartment 2, the gaseous constituent supply ECU 101 controls which gaseous constituent emitting unit is activated.

The navigation ECU 110 is a control unit of a navigation system that provides a fundamental function of at least indication of a present position, selection of a destination, calculation of a route, and route guide to the occupants 3, 4. The navigation ECU 110 also has a function of obtaining external information including VICS (Vehicle Information and Communication System; a registered trademark of Vehicle Information and Communication System Center), G-BOOK (contents service usable from an in-vehicle apparatus; a registered trademark of Toyota Motor Corporation), the Internet, etc. When this navigation ECU 110 obtains information that the road is congested from VICS or G-BOOK, it will send the information to the gaseous constituent supply ECU 101. In response to this, the gaseous constituent supply ECU 101 conducts a control that assumes that the occupants 3, 4, especially the driver, become sleepy, feeding a perfume constituent with the arousing effect reserved in the perfume constituent reserving means 17A as a gaseous constituent into the gaseous constituent supply chamber 15, and emitting it to the driver after making an air cannon projectile contain it. By this control, the driver breathes in the perfume constituent with the arousing effect etc. from the nose, whereby the driver can overcome sleepiness or obtain the arousing effect, which can prevent dozing off while driving.

Moreover, when the gaseous constituent supply ECU 101 obtains traffic congestion information from the navigation ECU 110, the gaseous constituent supply ECU 101 assumes that the traffic congestion has irritated the driver and other occupant, and conducts a control that feeds a perfume constituent with the relaxing effect that fills the perfume constituent reserving means 17B into the gaseous constituent supply chamber 15, and emitting an air cannon projectile containing the perfume constituent toward the driver.

Moreover, after the gaseous constituent supply ECU 101 emits the air cannon projectile containing a predetermined amount of the perfume constituent, it may halt emission of an air cannon projectile for a predetermined time or conduct an intermittent control that emits an air cannon projectile. Thus, by conducting an intermittent control under fixed conditions, the occupants 3, 4 can be urged to recover sense-of-smell from blunting of sense of smell caused by excess inhalation of the perfume constituent and become sensitive to even a small content of the perfume constituent.

The vehicular integrated ECU 120 has a function of acquiring working information of each functional component of the automobile 1 and sending it to the gaseous constituent supply ECU 101. Working information of the functional parts includes, for example, vehicle velocity information, braking information, vehicular gap sensor information, steering angle information of the steering wheel, window open/close information on an open/close state of the side window glass, sunroof open/close information on an open/close state of the sunroof, and the like. The vehicular integrated ECU 120 sends these pieces of information to the gaseous constituent supply ECU 101.

Note here that, the side window glass provided in the automobile 1 is designed to be capable of moving vertically by driving means that operates in response to a driving signal outputted from the vehicular integrated ECU 120, and this driving signal is generated according to an open/close operation by the driver with an operation switch. Therefore, when the driver performs an open operation on the operation switch, the vehicular integrated ECU 120 forms a driving signal that corresponds to the open operation and sends it to the driving means, whereby the side window glass is lowered to a predetermined position. On the other hand, when the driver performs a close operation on the operation switch, the vehicular integrated ECU 120 forms a driving signal that corresponds to the close operation and sends it to the driving means, whereby the side window glass is elevated to a predetermined position. The vehicular integrated ECU 120 determines whether the side window glass is in the close state or in the open state from the predetermined upper/lower position of the side window glass based on the above-mentioned driving signal, determines the open degree of the side window glass when it is in the open state, and outputs the result as the window open/close information to the gaseous constituent supply ECU 101. When the side window glass is in the uppermost position, the opening of the side window glass is determined to be 0%; when the side window glass is in the lowermost position, the opening of the side window glass is determined to be 100%. Since, two or more sheets of the side window glass capable of being moved vertically are provided, the vehicular integrated ECU 120 outputs the window open/close information regarding each side window glass to the gaseous constituent supply ECU 101.

Similarly, a sunroof provided to the automobile 1 is designed to be slidable in the front and rear direction by drive means operating based on a driving signal outputted from the vehicular integrated ECU 120. This driving signal is generated in response to the driver's open/close operation with the operation switch. Therefore, when the driver conducts the open operation on the operation switch, the vehicular integrated ECU 120 generates a driving signal that corresponds to the open operation and sends it to the drive means to implement sliding of the sunroof rearward to a predetermined position. On the other hand, when the driver conducts the close operation on the operation switch, the vehicular integrated ECU 120 generates a driving signal that corresponds to the close operation and sends it to the drive means to implement the sliding of the sunroof forward to a predetermined position. The vehicular integrated ECU 120 determines whether the sunroof is in the close state or in the open state from the front or rear position of it based on the above-mentioned driving signal, determines the open degree when it is in the open state, and outputs the result as the sunroof open/close information to the gaseous constituent supply ECU 101. When the sunroof is in a front-end position, the opening of the sunroof is determined to be 0%; when the sunroof is in a rear-end position, the opening is determined to be 100%.

When the gaseous constituent supply ECU 101 recognizes such pieces of information that the vehicle velocity is not more than a predetermined value, the number of times of breaking per unit time is not less than a predetermined number, a spacing to the preceding vehicle by the vehicular gap sensor is not more than a predetermined value, and the like, the ECU 101 considers the road is congested and performs both or either of a control that emits an air cannon projectile containing a perfume constituent with an arousing-effect reserved in the perfume constituent reserving means 17A toward the driver or a control that emits an air cannon projectile containing a perfume constituent with the relaxing effect reserved in the perfume constituent reserving means 17B as a gaseous constituent for giving the relaxing effect toward the driver.

Moreover, the gaseous constituent supply ECU 101 obtained a vehicle speed value of, for example, 80 km/h or more from vehicle speed information sent from the vehicular integrated ECU 120 continuously for a predetermined or more time, the gaseous constituent supply ECU 101 considers that the vehicle is traveling on a highway or the like and, in order to prevent the driver from dozing off while driving, conducts a control that feeds a perfume constituent with the arousing effect reserved in the perfume constituent reserving means 17A as a gaseous constituent into the gaseous constituent supply chamber 15, by a predetermined amount, and emits an air cannon projectile containing the perfume constituent toward the driver.

When the gaseous constituent supply ECU 101 determines that a steering direction of the steering device is varied to the right direction and to the left direction alternately and continuously based on steering angle information received from the vehicular integrated ECU 120, the gaseous constituent supply ECU 101 conducts a control that assumes that the vehicle travels a sequential curved road, such as a mountain pass, feeds a perfume constituent with the relaxing effect reserved in the perfume constituent reserving means 17C in order to alleviate a strained state into the gaseous constituent supply chamber 15, and emits an air cannon projectile containing the perfume constituent toward the driver. On the other hand, for other occupants, the gaseous constituent supply ECU 101 conducts a control that feeds a perfume constituent with a carsick preventive effect reserved in the perfume constituent reserving means 17C as a gaseous constituent for preventing carsickness to the gaseous constituent supply chamber 15, and emits an air cannon projectile containing the perfume constituent.

A biomedical sensor 180 detects biomedical signals of the occupants 3, 4, and sends the biomedical signals to the gaseous constituent supply ECU 101. The biomedical signals detected from the occupants 3, 4 include, for example, heart beat information such as the number of heat beats per unit time measured by a heart beat sensor, pupil information, such as sizes of pupils of the occupants 3, 4, brain-waves information of the occupants 3, 4, and the like. When the heart-beat information, the pupil information, and/or brain-waves information detected by the biomedical sensor 130 is information indicating that the occupants 3, 4 feel sleepy, the gaseous constituent supply ECU 101, as described above, selects a perfume constituent with the arousing effect among a plurality of different perfume constituents, feeds it into the gaseous constituent supply chamber 15, and emits an air cannon projectile containing that perfume constituent. On the other hand, when the information indicates that the occupants 3, 4 are in a strained state, the gaseous constituent supply ECU 101 selects a perfume constituent with the relaxing effect among a plurality of perfume constituents, feeds it in the gaseous constituent supply chamber 15, and emits an air cannon projectile containing that perfume constituent.

The plurality of infrared sensors 140 detect body temperatures of the occupants 3, 4 and send detection data constructed of the detection values to the gaseous constituent supply ECU 101. These infrared sensors 140 are held by posture alteration means (not shown in the figure) to operate based on control signals from the gaseous constituent supply ECU 101, and operations of the posture alteration means enable each infrared sensor 140 to direct its detecting direction to the face part and the upper half of the body of the occupants 3, 4. Among these infrared sensors 140, the infrared sensor 140 for detecting the body temperature of the front-seat occupant 3 can be installed in the instrument panel 70 and the infrared sensor 140 for detecting the body temperature of the rear-seat occupant 4 can be installed in a head rest 5A of the front seat (see FIG. 7). Not limited to this, each infrared sensor 140 may be installed in any position as long as the position allows the infrared sensor 140 to detect the body temperature of the occupants 3, 4.

The gaseous constituent supply ECU 101 determines whether the occupants 3, 4 are in a strained state or in a carsick state based on detection data received from the infrared sensor 140. When the detection value is higher than the body temperature at the time of normal temperature, the gaseous constituent supply ECU 101 determines that the occupants 3, 4 are in a strained state, selects a perfume constituent with the relaxing effect, feeds it into the gaseous constituent supply chamber 15, and emits an air cannon projectile containing that perfume constituent. On the other hand, when the detection value is lower than the body temperature at the time of normal temperature, the gaseous constituent-supply ECU 101 determines that the occupants 3, 4 are in a carsick state, selects a perfume constituent with a carsick prevention effect, feeds it into the gaseous constituent supply chamber 15, and emits an air cannon projectile containing that perfume constituent.

The air conditioner ECU 150 is designed to control an operation of each constituent element of the air conditioner, namely to do selecting a blowing mode and setting the air volume of the air-conditioning air being blown from an outlet port. Outlet port modes include: a DEF mode (defroster mode) in which air-conditioning air is blown at least from under lower side of the front wind shield glass and removes this wind shield glass fogging; a FACE mode in which air-conditioning air is blown to a direction of the face of the occupant from a center outlet port and a side face outlet port provided in the instrument panel 70; and a FOOT mode in which air-conditioning air is blown to the feet of the occupant 3. Moreover, as setup air volumes, the air conditioner ECU 150 is designed to be capable to set at least six stage air volumes of OFF, Lo, M1, M2, M3, and Hi in this order. Lo stands for a minimum air volume and Hi stands for a maximum air volume among the above-mentioned setup air volumes. In addition, the occupants 3, 4 can set arbitrarily a selection of the outlet port mode and the air volume with an operation panel whereby the occupants 3, 4 can operate setting. When the occupants 3, 4 select an automatic air conditioner function with the operation panel, the air conditioner ECU 150 can set these depending on a compartment environment. This air conditioner ECU 150 sends an air conditioner setting signal that indicates the occupant-selected (or ECU-selected) blowing mode and the setup air volume to the gaseous constituent supply ECU 101.

Moreover, the air conditioner with an in-vehicle swing mechanism for a swing louver is so designed that right or wrong of swinging of the swing louver can be set up by the occupants 3, 4 with the operation panel. Further, the air conditioner ECU 150 is designed to determine right or wrong of the swinging of the swing louver based on a compartment environment when the automatic air conditioning function is set up. Then, the air conditioner ECU 150 sends the air-conditioner setting signal in which information indicating a swing state of the swing louver is included to the gaseous constituent supply ECU 101.

The operation means 160 is means for, when the occupants 3, 4 want to have a perfume constituent by himself/herself, making the gaseous constituent emitting units 10-12 operate compulsively. For example, it is constructed in the form of a switch in a location where the occupants 3, 4 operate easily. The operation means 160 has at least an operation selection switch whereby an operation of the gaseous constituent emitting units 10-12 or operation halt can be selected and a perfume constituent selection switch whereby any one of a plurality of perfume constituents can be selected. This operation means 160 is designed to send a setting signal based on a setup item to the gaseous constituent supply ECU 101, and the gaseous constituent supply ECU 101 controls operations of the gaseous constituent emitting units 10-12 based on this.

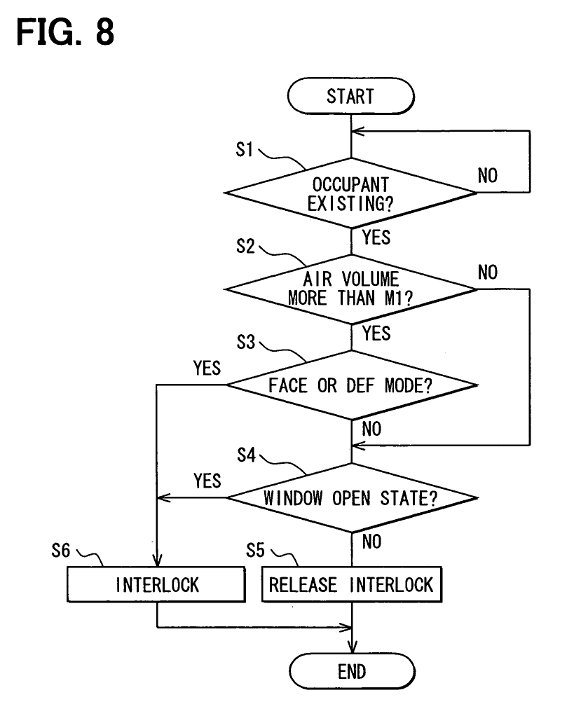

Moreover, the gaseous constituent supply ECU 101 controls operations of the gaseous constituent emitting units 10-12 based on a flowchart shown in FIG. 8 in response to the window open/close information from the vehicular integrated ECU 120 and a signal from the air conditioner ECU 150.

First, it is determined whether the occupants 3, 4 exist in the compartment 2 (Step S1). As information whereby the existence of the occupants 3, 4 is determined, for example, there is an on/off state of an ignition switch or a detection value by the infrared sensor 140. For example, when the ignition switch is turned on or when the detection value of the infrared sensor 140 substantially coincides with the human body temperature, it can be determined that the occupants 3, 4 exist.

When it is determined that the occupants 3, 4 exist (YES in Step S1), the gaseous constituent supply ECU 101 receives an air conditioner setting signal from the air conditioner ECU 150, and also receives the window open/close information and the sunroof open/close information from the vehicular integrated ECU 120. Among various states with the existence of the occupants 3, 4, when the state is either a case where the setup air volume of the air-conditioning air is M1 or more and the blowing mode indicates the FACE mode or the DEF mode (YES in Step S2 and in Step S3), or a case where the window open/close information indicates an open state (YES in Step S4), the gaseous constituent supply ECU 101 infers that it is the airflow generation state in which an airflow is generated in the compartment 2, it halts (interlock) operations of the gaseous constituent emitting units 10-12 (Step S6). The reason of controlling in this manner is to prevent the following faults: an air cannon projectile is diverted from an occupant direction by airflows, such as an air-conditioning air and an external airflow invading from the side window glass, and emitted toward an occupant who does not need it; an air cannon projectile is diffused before reaching the occupants 3, 4; and the like.

On the contrary, when the setup air volume indicates M1 or less (NO in Step S2) and the selection signal indicates a mode other than the FACE mode and the DEF mode (NO in Step S3), the gaseous constituent supply ECU 101 infers that it is a non-airflow state in which no airflow is generated in the compartment and permits operations of the gaseous constituent emitting units 10-12 (releases interlock) (Step S5). Moreover, when the window open/close information indicates the close state or the sunroof open/close information indicates the close state (NO in Step S4), the gaseous constituent supply ECU 101 infers that it is the non-airflow state, like the above, in which no airflow is generated in the compartment 2 and permits operations of the gaseous constituent emitting units 10-12 (releases interlock) (Step S5). In this way, the gaseous constituent supply ECU 101 conducts the above-mentioned operation control to the gaseous constituent emitting units 10-12.

Moreover, when information indicating a side window open state is obtained from the vehicular integrated ECU 120, the gaseous constituent supply ECU 101 may determine right or wrong of the interlock based on the open degree of it; for example, when the open degree of the side window is indicated 50% or more, the above-mentioned interlock is performed, while, when the open degree is indicated 50% or less, interlock is released. The open degree whereby right or wrong of the interlock is determined can be set to various values other than the above-mentioned 50%.

Moreover, right or wrong of interlock may be determined based on an open/close state of each side window glass. For example, when two sheets of side window glass facing each other is set to be in an open state, interlock shall be performed. Specifically, two sheets of side window glass are arranged in the front seat and in the rear seat, respectively, when the side window glass arranged to at least one of the front seat side and the rear seat side is indicated to be in the open state, interlock will be performed. Usually, when the two sheets of the side window glass facing each other are set in the open state, an airflow flowing in a direction perpendicular to a traveling direction of the automobile becomes easy to invade into the compartment 2. Therefore, by conducting the control in the above manner, this gaseous constituent can certainly be supplied to the occupants 3, 4 who want to have this gaseous constituent, while preventing the following faults: an air cannon projectile is diverted for an occupant direction by an external airflow invading from the side window glass and the like and emitted toward an occupant who does not need it; an air cannon projectile is diffused before reaching the occupants 3, 4; and the like. Determination of right and wrong of the above-mentioned interlock may be done in a compound manner.

Moreover, when the information indicating a sunroof open state is obtained from the vehicular integrated ECU 120, right and wrong of interlock may be determined based on the open degree; for example, when the open degree of the sunroof is indicated 50% or more, the above-mentioned interlock is performed, while the open degree is indicated 50% or less, interlock is released. The open degree whereby right or wrong of interlock is determined can be set to various values other than the above-mentioned 50%.

Moreover, by doing as follows, operations of the gaseous constituent emitting units 10-12 can also be controlled. The gaseous constituent supply ECU 101 sends a control signal to each posture alteration means and turn the infrared sensor 140 to a predetermined area including the face parts and half of the bodies of the occupants 3, 4 by altering the posture of the infrared sensor 140, and makes the infrared sensor 140 scan temperatures in each detection direction. Then, the gaseous constituent supply ECU 101 receives a detection value in the each detection direction of the sensor 140, and detects seating states of the occupants 3, 4 based on detection data constructed of these detection values. This operation is performed at predetermined intervals to detect a change of the seating states of the occupants 3, 4. Note here that, when the occupants 3, 4 move the upper half of the bodies largely in order to alter seating positions of the occupants 3, 4, for example, the gaseous constituent supply ECU 101 detects that seating states of the occupants 3, 4 have been changed, and infers that an airflow is generated in the compartment 2 by this, and halts operations of the gaseous constituent emitting units 10-12 (interlock). On the other hand, when there is no change in the seating states of the occupants 3, 4, the gaseous constituent supply ECU 101 infers that it is a non-airflow sate in which no airflow is generated in the compartment 2 and permits operations of the gaseous constituent emitting units 10-12 (releases interlock).

In this embodiment, the gaseous constituent supply ECU 101 is configured to emit an air cannon projectile containing the perfume constituent toward the occupants 3, 4 in this way, an optimum perfume constituent can be supplied to each of the occupants 3, 4. Moreover, when the gaseous constituent supply ECU 101 infers that it is the non-airflow state in which no airflow is generated in the compartment 2, operations of the gaseous constituent emitting units 10-12 are permitted, while, when the gaseous constituent supply ECU 101 infers that it is an airflow state in which an airflow is generated, the operations of the gaseous constituent emitting units 10-12 are halted. Therefore, supply of a perfume constituent to the occupants 3, 4 is not hampered, and the perfume constituent can certainly be supplied.

Moreover, two or more kinds of perfume constituents are reserved using the perfume constituent reserving means 17A-17C, and an optimum perfume constituent is selected based on a traveling state of the automobile 1 and the body conditions of the occupants 3, 4, and these are supplied to the occupants 3, 4, whereby comfortable states of the respective occupants 3, 4 can be improved.

Moreover, the operation means 160 is provided, and the occupants 3, 4 are enabled to select the operations of the gaseous constituent emitting units 10-12 and the operation halt and select a perfume constituent. By this setting, the gaseous constituent supply ECU 101 can supply an optimum perfume constituent in a timing that the occupants 3, 4 desire.

### Second Embodiment

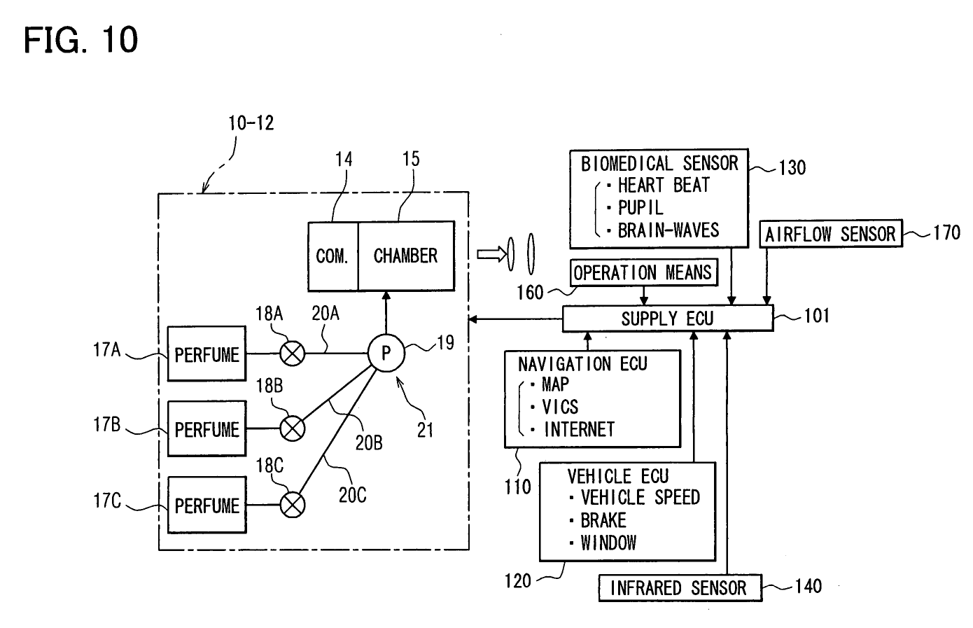

The second embodiment according to this invention will be described with reference to FIGS. 9 and 10. The same parts as those of the above-mentioned first embodiment will be designated the same reference numerals and redundant description thereof will be omitted. Description of the same operations and effects will also be emitted, and only differences will be described.

In this embodiment, a plurality of airflow sensors 170 each for detecting an airflow are provided in the compartment 2, and operations of the gaseous constituent emitting units 10-12 are controlled based on detection values of these airflow sensors 170.

The airflow sensors 170 are arranged to be able to detect airflows in the space between the gaseous constituent emitting units 10-12 and the occupants 3, 4. For example, the airflow sensor 170 is installed on the ceiling part in the compartment 2. The airflow sensors 170 each output a detection value that corresponds to the detected flow velocity of the airflow to the gaseous constituent supply ECU 101.

The gaseous constituent supply ECU 101 controls operations of the gaseous constituent emitting units 10-12 based on the detection values received from the airflow sensors 170. Specifically, when the detection value detected by each airflow sensor 170 is found to be not more than a predetermined value, operations of the gaseous constituent emitting units 10-12 are permitted because the emitted air cannon projectile can certainly be supplied to an occupant who wants it.

On the other hand, when the detection value of the airflow sensor 170 for detecting an airflow in a space between the gaseous constituent emitting units 10, 11 and the occupant 3 is found to be not less than a predetermined value, operations of the gaseous constituent emitting units 10, 11 are halted because there is the possibility that the air cannon projectile may be emitted toward other occupant after being diverted from an occupant direction by the airflow or may be diffused before reaching the occupant 3. When the detection value of the airflow sensor 170 for detecting an airflow in a space between the gaseous constituent emitting unit 12 and the occupant 4 is found to be not less than a predetermined value, an operation of the gaseous constituent emitting unit 12 is halted because of the same reason as described above.

In this embodiment, the gaseous constituent supply ECU 101 is configured to control operations of the gaseous constituent emitting units 10-12 based on detection values of the airflow sensors 170. When the ECU 101 is configured in this way, supply of the air cannon projectile is not hampered depending on the airflow state, and the gaseous constituent can surely be supplied to the occupants 3,4, while being able to resolve a fault in which emitted gaseous constituent is supplied to an occupant who does not need it.

### Third Embodiment

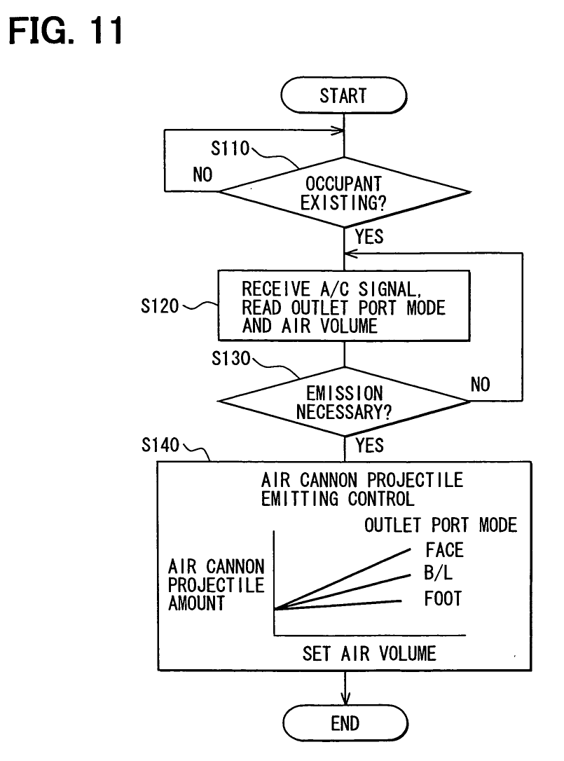

The third embodiment according to this invention will be described with reference to FIG. 11. Description of the same parts as those of the above-mentioned first embodiment will be omitted, and only differences will be described. This embodiment is different from the above-mentioned first embodiment in that the gaseous constituent emitting units 10-12 are controlled based on an operating state of the air conditioner so that an emitted air cannon projectile may reach the occupants 3,4.

As shown in FIG. 11, the gaseous constituent supply ECU 101 determines first whether the occupants 3, 4 exist in the compartment 2 (Step S110). A method for determining the existence of the occupants 3, 4 is the same as that of the above-mentioned first embodiment. When it is determined that the occupants 3, 4 exist (YES in Step S110), the ECU 101 receives the air-conditioner setting signal from the air conditioner ECU 150 and grasps the outlet port mode and a setup air volume as an operating state of the air conditioner (Step S120).

Next, it is determined whether it is necessary to emit an air cannon projectile to the occupants (Step S130), like the above-mentioned first embodiment. When the emission is necessary (YES in Step S130), the airflow state in the compartment 2 is inferred based on the outlet port mode and the setup air volume, and the gaseous constituent emitting units 10-12 are so controlled that emitted air cannon projectiles may reach the occupants 3, 4 (Step S140).

Specifically, as shown in Step S140 in FIG. 11, in order to supply a predetermined amount of a gaseous constituent, an air volume of the air cannon projectile is determined according to a setup air volume in the setup outlet port, and the gaseous constituent emitting units 10-12 are so controlled that the air cannon projectile may have the determined air volume. Note here that, the air volume of an air cannon projectile has a linear relationship with the setup air volume of the air-conditioning air. This is because, as the setup air volume of the air-conditioning air increases, airflows generated in the compartment 2 increase and it becomes difficult for the air cannon projectile to reach the occupants 3, 4; therefore, it becomes necessary to emit an air cannon projectile with an air volume that overcomes the generated airflows.

Moreover, the air volumes in the outlet port modes are determined in such a way that the air volume increases in the order of the FOOT mode, the B/L mode, and the FACE mode. The reason is that it is necessary to set the air volume of an air cannon projectile comparatively largely in the outlet port mode where the ratio of the air volume of the air-conditioning air flowing toward the face parts of the occupants 3, 4 is large because, when the air-conditioning air flowing toward the face parts of the occupants 3, 4 increases, the air cannon projectile becomes easy to be diffused and becomes difficult to reach the occupant 3,4.

When the air volume is altered depending on the outlet port mode of the air-conditioning air and the setup air volume, it is necessary just to alter, for example, the flow velocity of an air cannon projectile and the number of emissions of an air cannon projectile (emission period).

Note here that, when the determined air volume of the air cannon projectile increases, it implies that the air volume of the air-conditioning air blowing to the face parts of the occupants 3, 4 increases, and consequently it becomes necessary to emit an air cannon projectile that would not be diffused by this air-conditioning air. Therefore, when the air volume of the air cannon projectile is intended to be increased, it is preferable that the flow velocity of the air cannon projectile is increased first and then the number of emissions is increased. Note that in the case where the flow velocity of the air cannon projectile does not reach the desired flow velocity due to performance of the air compression means 14 etc., some of air cannon projectiles may be diffused halfway by the air-conditioning air. In such a case, what is necessary is to increase both or either of the number of emissions of air cannon projectiles and the concentration of a gaseous constituent that is contained in an air cannon projectile, and consequently to increase the amount of supply of the gaseous constituent to be supplied to the occupants 3, 4.

According to this embodiment, since an air cannon projectile can be supplied to the occupants 3, 4 even when it is in an airflow generation state due to an operating state of the air conditioner, the gaseous constituent can be supplied to the occupants 3, 4 when needed, without depending on the airflow state. Moreover, it can be inferred whether the air-conditioning air generates an airflow in the compartment 2. Furthermore, when it is in the airflow generation state, in which an airflow is being generated, a flowing direction of the airflow and its air volume can also be inferred; therefore, the probability that air cannon projectiles emitted from the gaseous constituent emitting units 10-12 will reach the occupants 3, 4 can be increased.

### Fourth Embodiment

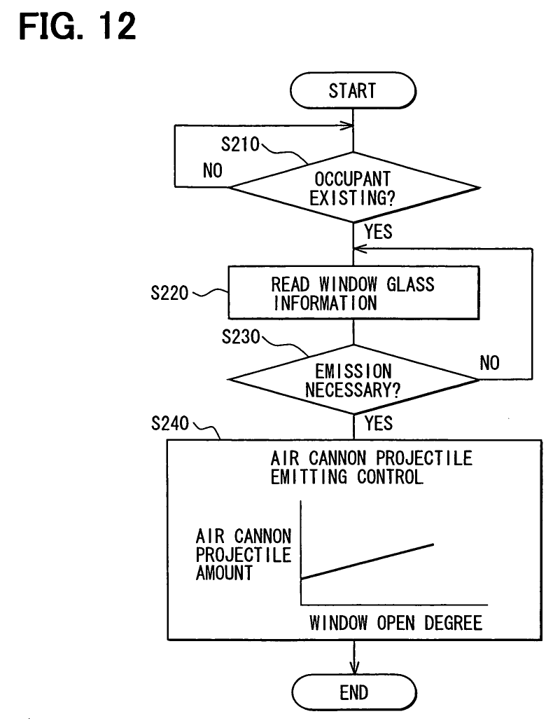

The fourth embodiment according to this invention will be described with reference to FIG. 12. Description of the same parts as those of the above-mentioned first embodiment will be omitted, and only differences will be described. This embodiment differs from the above-mentioned first embodiment in that, based on the open/close state of the side window glass, the gaseous constituent emitting units 10-12 are so controlled that the emitted air cannon projectile will reach the occupants 3, 4.

As shown in FIG. 12, the gaseous constituent supply ECU 101 determines first whether the occupants 3, 4 exist in the compartment 2 (Step S210). A method for determining the existence of the occupants 3, 4 is the same as that of the above-mentioned first embodiment. When the ECU 101 determines the existence of the occupants 3, 4 (YES in Step S210), it receives the window open/close information from the vehicular integrated ECU 120 and grasps the open degree of the side window glass (Step S220).

Next, the ECU 101 determines whether it is necessary to emit an air cannon projectile (Step S230), like the first embodiment. When the emission is necessary (YES in Step S230), the airflow state in the compartment 2 is inferred based on the open degree of the side window glass, and the gaseous constituent emitting units 10-12 are so controlled that the air cannon projectile being emitted may reach the occupants 3, 4 (Step S240).

Specifically, as shown in Step S240 in FIG. 12, the air volume of the air cannon projectile is determined according to the open degree of the side window glass so that a predetermined amount of a gaseous constituent should be supplied, and the gaseous constituent emitting units 10-12 are so controlled that each air volume may become equal to the determined air volume. Note here that the air volume of the air cannon projectile has a proportional relationship with the open degree of the side window glass. This is because, with increasing open degree of the side window glass, the external airflow invading into the compartment 2 increases and the air cannon projectile becomes difficult to reach the occupants 3, 4; therefore, it becomes necessary to emit an air cannon projectile with such an air volume that would overcome the generated airflow.

In the case where the air volume of the air cannon projectile is altered according to the open degree of the side window glass, what is necessary is, for example, just to alter the air volume of the air cannon projectile and the number of emissions of the air cannon projectile (emission period).

When the determined air flow of the air cannon projectile increases, it implies that the air volume of the external airflow invading into the compartment 2 increases, and consequently it becomes necessary to emit an air cannon projectile that would not be diffused by this external airflow. Therefore, it is preferable that, when the air volume of the air cannon projectile is intended to be increased, the flow velocity of the air cannon projectile is increased first and then the number of emissions is increased. In the case where the flow velocity of the air cannon projectile does not become equal to a desired flow velocity due to performance of the air compression means 14 etc., some of emitted air cannon projectiles are diffused halfway by the air-conditioning air. In such a case, by increasing both or either of the number of emissions of the air cannon projectile and the concentration of the gaseous constituent that is contained in an air cannon projectile, it is possible to increase the amount of supply of a gaseous constituent to be supplied to the occupants 3, 4.

According to this embodiment, since even when it is in the airflow generation state caused by invasion of the external airflow through the side window glass, an air cannon projectile can be supplied to the occupants 3, 4, the gaseous constituent can be supplied to the occupants 3, 4 without depending on the airflow state. Moreover, it can be inferred whether it is in the airflow generation state caused by invasion of the external airflow from the open degree of the side window glass, and further an air volume of the external airflow can be inferred. Therefore, by controlling the gaseous constituent emitting units 10-12 based on this, the probability that an air cannon projectile will reach the occupants 3, 4 can be increased.

### Fifth Embodiment

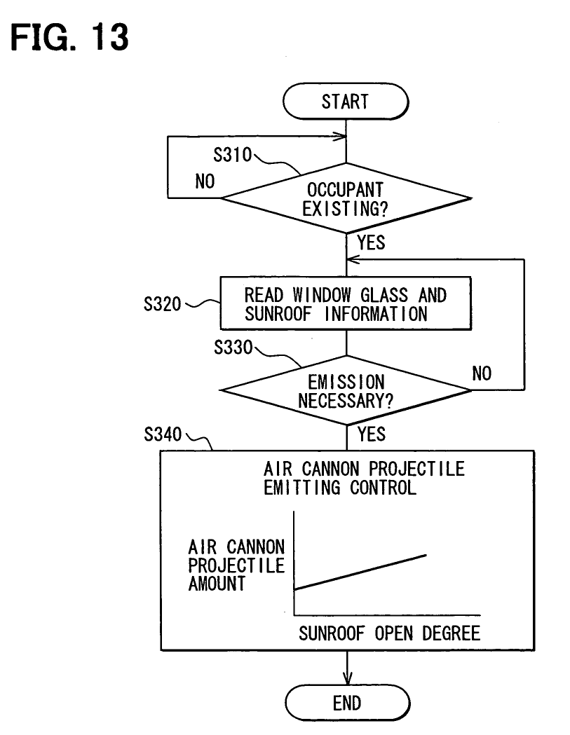

The fifth embodiment according to this invention will be described with reference to FIG. 13. Description of the same parts as those of the above-mentioned first embodiment will be omitted, and only differences will be described. This embodiment differs from the above-mentioned first embodiment in that the gaseous constituent emitting units 10-12 are controlled based on the open/close state of the sunroof so that an emitted air cannon projectile may reach the occupants 3, 4.

As shown in FIG. 13, the gaseous constituent supply ECU 101 determines first whether the occupants 3, 4 exist in the compartment 2 (Step S310). A method for determining the existence of the occupants 3, 4 is the same as that of the above-mentioned first embodiment. When it is determined that the occupants 3, 4 exist (YES in Step S310), the gaseous constituent supply ECU 101 receives the open/close information of the sunroof from the vehicular integrated ECU 120 and grasps the open degree of the sunroof (Step S320).

Next, it is determined whether it is necessary to emit an air cannon projectile (Step S330), like the above-mentioned first embodiment. When the emission is necessary (YES in Step S330), the airflow state in the compartment 2 is inferred based on the open degree of the sunroof, and the gaseous constituent emitting units 10-12 are so controlled that an emitted air cannon projectile may reach the occupants 3, 4 (Step S340).

Specifically, as shown in Step S340 in FIG. 13, the air volume of the air cannon projectile is determined according to the open degree of the sunroof so that a predetermined amount of a gaseous constituent should be supplied, and the gaseous constituent emitting units 10-12 are so controlled that each air volume may become equal to the determined air volume. Note here that the air volume of the air cannon projectile has a proportional relationship with the open degree of the sunroof. The reason is that, with increasing open degree of the sunroof, the external airflow invading into in the compartment 2 also increases and the air cannon projectile becomes difficult to reach the occupants 3, 4, and consequently it becomes necessary to emit an air cannon projectile with such an air volume that would overcome the generated airflow.

In the case where the air volume of the air cannon projectile is altered according to the open degree of the sunroof, what is necessary is, for example, just to alter the flow velocity of the air cannon projectile and the number of emissions of the air cannon projectile (emission period).

Note here that, when the determined air volume of the air cannon projectile increases, it implies that the air volume of the external airflow invading into the compartment 2 increases, and consequently it becomes necessary to emit an air cannon projectile that would not be diffused by this external airflow. Therefore, when the air volume of the air cannon projectile is intended to be increased, it is preferable that the flow velocity of the air cannon projectile is increased first and then the number of emissions is increased. Note that in the case where the flow velocity of the air cannon projectile does not become equal to a desired flow velocity due to performance of the air compression means 14 etc., some of emitted air cannon projectiles may be diffused halfway by the air-conditioning air. In such a case, what is necessary is to increase both or either of the number of emissions of the air cannon projectile and the concentration of the gaseous constituent that is contained in an air cannon projectile, and consequently to increase the amount of supply of the gaseous constituent to be supplied to the occupants 3, 4.

According to this embodiment, since even in the airflow generation state in which an airflow is generated by an external airflow invading from the sunroof, an air cannon projectile can be supplied to the occupants 3, 4, a gaseous constituent can be supplied to the occupants 3, 4 when needed, not depending on the airflow state. Moreover, it can be inferred whether an external airflow invades and causes the airflow generation state by the open degree of the sunroof, and the amount of the external airflow that invades can be inferred. Therefore, by controlling the gaseous constituent emitting units 10-12 based on this, the probability of the air cannon projectile reaching the occupants 3, 4 can be increased.

### Sixth Embodiment

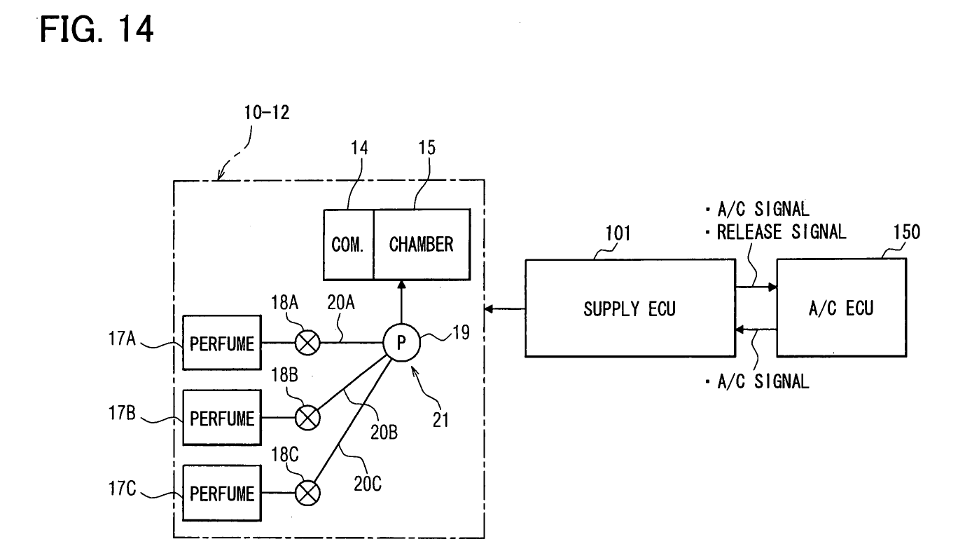

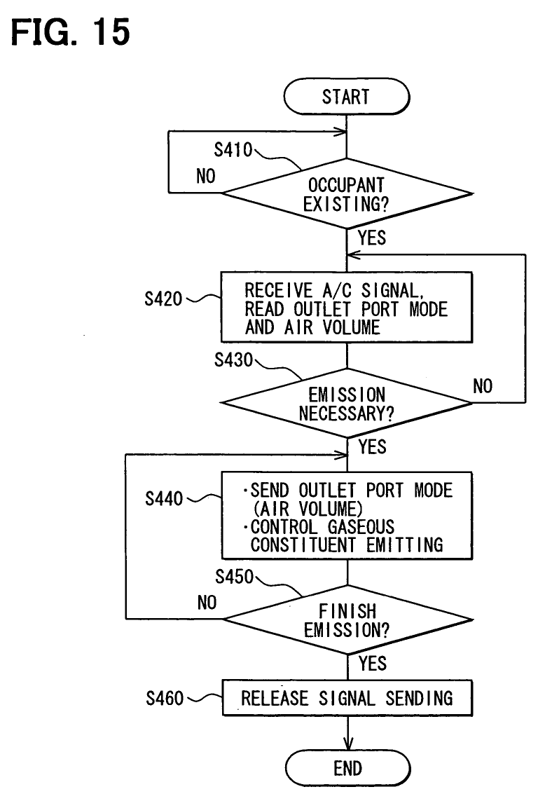

The sixth embodiment according to this invention will be described with reference to FIGS. 14 and 15. Description of the same parts as those of the above-mentioned first embodiment will be omitted, and only differences will be described. This embodiment differs from the first embodiment in that, when emitting an air cannon projectile, the air conditioner is controlled in order to reduce or eliminate an airflow generated by the air-conditioning air.

The gaseous constituent supply ECU 101 (supply ECU) is designed to be capable of receiving the air conditioner setting signal from the air conditioner ECU 150 (A/C ECU), as shown in FIG. 14, and further to be capable of selectively sending an air conditioner control signal for setting up an outlet port mode, a setup air volume, and swinging of the swing louver, and a release signal used to release the settings by that air conditioner control signal to the vehicular integrated ECU 120.

On the other hand, while the air conditioner ECU 150 receives an air conditioner control signal from the gaseous constituent supply ECU 101, the ECU 150 controls the outlet port mode, the setup air volume, and the swinging of the swing louver based on this signal.

First, the gaseous constituent supply ECU 101 determines whether the occupants 3, 4 exist in the compartment 2 (Step S410), as shown in FIG. 15. A method for determining the existence of the occupants 3, 4 is the same as that of the above-mentioned first embodiment. When the ECU 101 determines that the occupants 3, 4 exist (YES in Step S410), the ECU 101 receives the air conditioner setting signal from the air conditioner ECU 150 and grasps the outlet port mode, the air volume, and the swing state of the swing louver (Step S420).

Next, the ECU 101 determines whether it is necessary to emit an air cannon projectile (Step S430), like the above-mentioned first embodiment. When the emission is necessary (YES in Step S430), the ECU 101 controls the outlet port mode, the setup air volume, and swinging of the swing louver based on a current outlet port, setup air volume, and a swing state of the swing louver of the air-conditioning air so that the air-conditioning air may not diffuse an air cannon projectile, and controls operations of the gaseous constituent emitting units 10-12 to perform the emission of the air cannon projectile (Step S440).

Specifically, when the setup air volume is not less than M1 and the blowing mode is set to the FACE mode, it is inferred that a state is the airflow generation state in which the air-conditioning air generates an airflow in the face part and its vicinity of the occupants 3, 4, and the ECU 101 sends, to the air conditioner ECU 150, an air conditioner control signal for setting the setup air volume to Lo and the outlet port mode to the FOOT mode, and makes the gaseous constituent emitting units 10-12 operate.

In response to these operations, the air conditioner ECU 150 having received the air conditioner control signal (A/C signal) will change the current setup air volume M1 to Lo and switch the outlet port mode from the FACE mode to the FOOT mode. By this, the air-conditioning air is reduced in air volume and is directed to a direction different from the face parts of the occupants 3, 4, and accordingly the airflow generated in the face parts and its vicinity of the occupants 3, 4 will disappear or reduce. Therefore, the air cannon projectiles emitted from the gaseous constituent emitting units 10-12 reach the occupants 3, 4 without being diffused halfway.

When the emission of the air cannon projectile is finished (Yes in Step S450), the gaseous constituent supply ECU 101 sends the release signal to the air conditioner ECU 150 (Step S460). Then, the air conditioner ECU 150 releases the outlet port mode and the setup air flow that were set up by the air conditioner control signal, and sets up an outlet port mode and an air volume based on a setting of an operator panel or an automatic air-conditioning function.

The control of an operating state of the above-mentioned air conditioner is executed by sending the air conditioner control signal when the outlet port mode and the setup air volume are set to predetermined setting. However, the air conditioner control signal may be sent paying attention to either only the setup air volume or only the outlet port mode. For example, when the setup air volume is set to not less than M1, an air conditioner control signal for setting this setup air volume to Lo or OFF is sent. Moreover, when the outlet port mode is set to the FACE mode, an air conditioner control signal for setting this mode to the FOOT mode or B/L mode (bi-level mode) is sent.

Furthermore, when the swing louver is swinging, air conditioner control signal for halting the swinging of the swing louver toward the occupants 3, 4 and fixing a swing position of the swing louver in a different direction from that of the occupants 3, 4 may be sent.

Moreover, as a control of the operating state of the air conditioner, a control may be done as follows. That is, when emitting an air cannon projectile, the outlet port mode is set in such a way that its flow velocity becomes equal to the flow velocity of the air cannon projectile. By this setting, the air cannon projectiles emitted from the gaseous constituent emitting units 10 disposed in the instrument panel 70 among the air cannon projectiles emitted from the gaseous constituent emitting units 10-12 reach the occupants 3, 4 without being diffused by the air-conditioning air because their flow velocities become substantially the same as that of the air-conditioning air, being in substantially parallel direction to a flowing direction of the air-conditioning air.

Moreover, a time difference until the air conditioner establishes the predetermined airflow state or eliminates/reduces an airflow in the compartment 2 after the start of control of the air conditioner may take place. When the emission timing of the air cannon projectile is set to the same time as the control timing of the air conditioner, an air cannon projectile will be emitted while an airflow continues to be generated in the compartment 2, and consequently this air cannon projectile may be diffused. Therefore, it is preferable that the timing to control the air conditioner is set earlier than the control timing of the gaseous constituent emitting units 10-12, i.e., the emission timing of the air cannon projectile.

According to this embodiment, since the air conditioner is so controlled that airflows generated by the air-conditioning air may be eliminated or reduced, the gaseous constituent can be supplied to the occupants 3, 4 when needed. Moreover, since the outlet port mode and the setup air volume of the air-conditioning air are so controlled that the air cannon projectile may not be diffused, even when the state is set to the airflow generation state, a gaseous constituent can be supplied to the occupants 3, 4 when needed.

### Seventh Embodiment

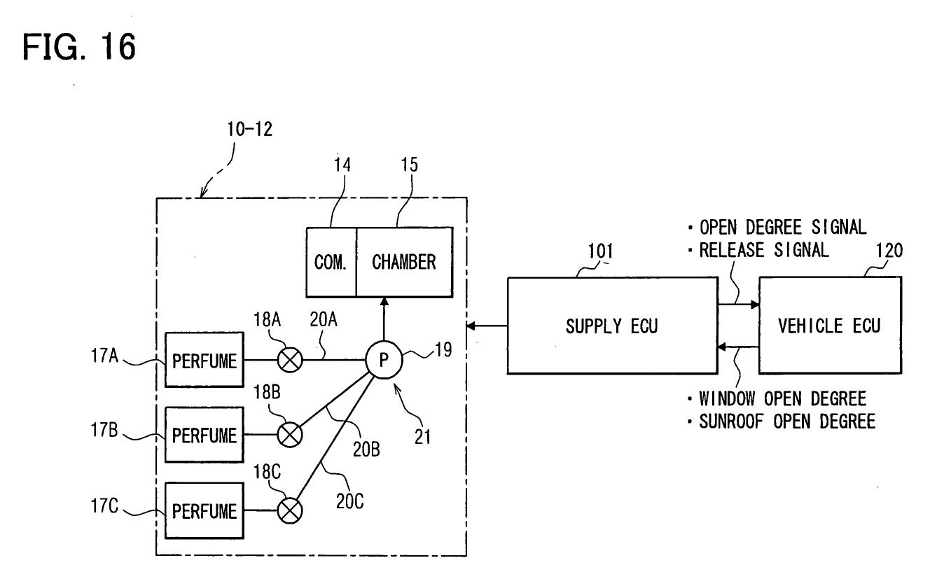

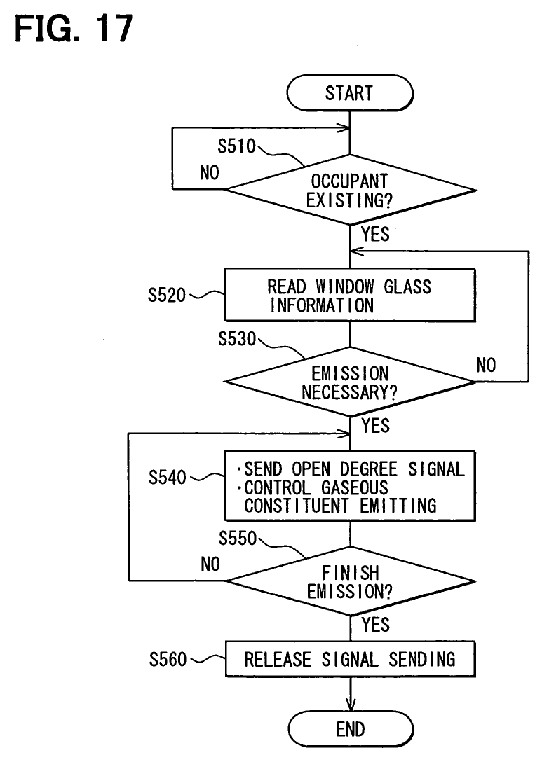

The seventh embodiment according to this invention will be described with reference to FIGS. 16 and 17. Description of the same parts as those of the above-mentioned first embodiment will be omitted, and only differences will be described. This embodiment differs from the first embodiment in that, when an air cannon projectile is emitted, opening of side window glass is controlled in order to reduce or eliminate external airflow invading into the compartment 2 through the side window glass.

The gaseous constituent supply ECU 101 is designed to be capable of receiving the window open/close information from the vehicular integrated ECU 120, as shown in FIG. 16, and further to be capable of selectively sending an open degree specification signal used to specify the open degree of the side window glass and a release signal used to release the specification of the open degree of the side window glass based on this open degree specification signal to the vehicular integrated ECU 120.

On the other hand, when the vehicular integrated ECU 120 (vehicle ECU) receives the open degree specification signal from the gaseous constituent supply ECU 101, the vehicular integrated ECU 120 sets the open degree of the side window glass to a specified open degree based on this signal.

First, the gaseous constituent supply ECU 101 determines, as shown in FIG. 17, whether the occupants 3, 4 exist in the compartment 2 (Step S510). A method for determining the existence of the occupants 3, 4 is the same as that of the above-mentioned first embodiment. When it is determined that the occupants 3, 4 exist (YES in Step S510), the gaseous constituent supply ECU 101 receives the window open/close information from the vehicular integrated ECU 120 and grasps and reads the open degree of the side window glass (Step S520).

Next, the ECU 101 determines whether it is necessary to emit an air cannon projectile (Step S530), like the above-mentioned first embodiment. When it is necessary to emit the air cannon projectile (YES in Step S530), the ECU 101 controls the open degree of the side window glass based on the open degree of the side window glass so that the air cannon projectile is not diffused by an external airflow invading from the outside (Step S540). For a control of the open degree of the side window glass, for example, when the open degree of the side window glass is 0% or more, an open degree specification signal for specifying the open degree as 0% is sent and the gaseous constituent emitting units 10-12 are operated.

Then, the vehicular integrated ECU 120 that has received the open degree specification signal sets up the open degree of the side window glass to 0%. By this setting, the side window glass becomes a close state, and accordingly an external airflow is prevented from invading into the compartment 2. Therefore, air cannon projectiles emitted from the gaseous constituent emitting units 10-12 reach the occupants 3, 4, without being diffused by the external airflow.

When the emission of the air cannon projectile is finished (YES in Step S550), the gaseous constituent supply ECU 101 sends the release signal to the vehicular integrated ECU 120 (Step S560). Then, the vehicular integrated ECU 120 releases the open degree of the side window glass specified by the open degree specification signal, and sets it at the open degree before receiving the open degree specification signal.

The open degree of the side window glass may be controlled to be other than 0%, for example, 20%, 30%, etc. Moreover, the ECU 120 may be configured to set the open degree of the side window glass based on vehicle velocity information received from the vehicular integrated ECU 120. The reason is that the open degree of the side window glass is reduced with increasing vehicle velocity, whereby the invasion quantity of the external airflow is suppressed because the external airflow invading the compartment 2 increases as the vehicle velocity increases. For example, when the vehicle velocity is 40 km/h, the open degree of the side window glass is controlled to be 20%; when the vehicle velocity is 80 km/h, the open degree of the side window glass is controlled to be 10%.

Moreover, a time difference may take place until an airflow in the compartment 2 is eliminated or reduced after the start of control of the open degree of the side window glass. When the emission timing of the air cannon projectile is set to the same time as a control timing of the air conditioner, an air cannon projectile will be emitted while an airflow continues to be generated in the compartment 2, and consequently this air cannon projectile may be diffused. Therefore, it is preferable that a timing in which the open degree of the side window glass is controlled is set earlier than the control timing of the gaseous constituent emitting units 10-12, i.e., the emission timing of the air cannon projectile.

According to this embodiment, because the open degree of the side window glass is so controlled that the external airflow invading at the time of emission of the air cannon projectile is eliminated or reduced, a gaseous constituent can be supplied to the occupants 3, 4 when needed.

### Eighth Embodiment

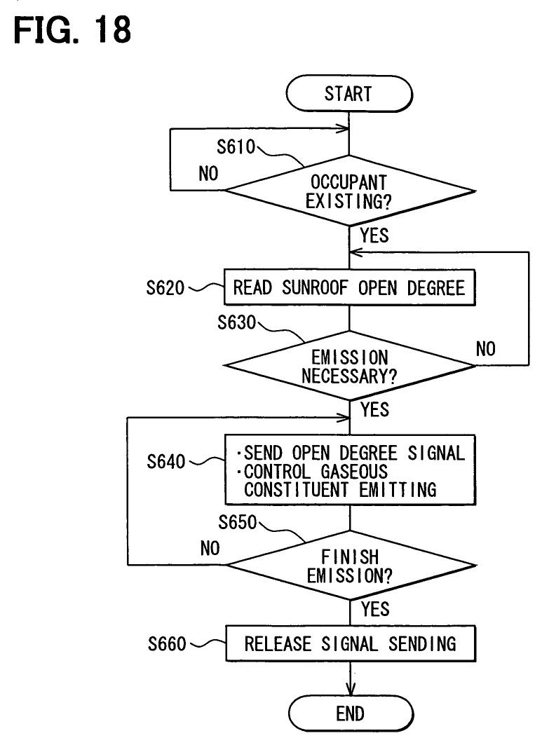

The eighth embodiment according to this invention will be described with reference to FIGS. 16 and 18. Description of the same parts as those of the above-mentioned first embodiment will be omitted, and only differences will be described. This embodiment differs from the first embodiment in that, when emitting an air cannon projectile, the open degree of a sunroof is controlled in order to reduce or eliminate an external airflow that invades into the compartment 2 through the sunroof.

The gaseous constituent supply ECU 101 is designed to be capable of receiving the sunroof open/close information from the vehicular integrated ECU 120, as shown in FIG. 16, and further to be capable of selectively sending an open degree specification signal used to specify the open degree of the sunroof and a release signal used to release the specification of the open degree of the sunroof based on this open degree specification signal, to the vehicular integrated ECU 120.

On the other hand, when the vehicular integrated ECU 120 receives the open degree specification signal from the gaseous constituent supply ECU 101, the ECU 120 sets the open degree of the sunroof to the specified open degree based on this signal.

First, the gaseous constituent supply ECU 101 determines whether the occupants 3, 4 exist in the compartment 2 (Step S610), as shown in FIG. 18. A method for determining the existence of the occupants 3, 4 is the same as that of the above-mentioned embodiments. When the ECU 101 determines that the occupants 3, 4 exist (YES in Step S610), it receives the sunroof open/close information from the vehicular integrated ECU 120 and grasps and reads the open degree of the sunroof (Step S620).

Next, the ECU 101 determines whether it is necessary to emit an air cannon projectile (Step S630), like the above-mentioned first embodiment. When the emission is necessary (YES in Step S630), the ECU 101 controls the open degree of the sunroof based on the specified open degree of the sunroof so that an external airflow invading from the outside may not diffuse the air cannon projectile (Step S640). Regarding a control of the open degree of the sunroof, for example, when the open degree of the sunroof is 0% or more, an open degree specification signal to specify the open degree as 0% is sent and the gaseous constituent emitting units 10-12 are operated.

In response to this, the vehicular integrated ECU 120 that receives the open degree specification signal will set the open degree of the sunroof to 0%. By this setting, since the sunroof will be in a close state, an external airflow is prevented from invading into the compartment 2. Therefore, air cannon projectiles emitted from the gaseous constituent emitting units 10-12 reach the occupants 3, 4 without being diffused by an external airflow.

When the emission of the air cannon projectile is finished (YES in Step S650), the gaseous constituent supply ECU 101 sends the release signal to the vehicular integrated ECU 120 (Step S660). Then, the vehicular integrated ECU 120 releases the open degree of the sunroof specified by the open degree specification signal, and sets it to the open degree before receiving the open degree specification signal.

The open degree of the sunroof may be controlled to be other than 0%, for example, 20%, 30%, etc. Alternatively, the open degree of the sunroof may be set up based on vehicle velocity information sent from the vehicular integrated ECU 120. The reason is that the open degree of the sunroof is reduced with increasing vehicle velocity, whereby the invasion quantity of the external airflow is suppressed because the external airflow invading the compartment 2 increases with increasing vehicle velocity. For example, when the vehicle velocity is 40 km/h, the open degree of the sunroof is controlled to be 20%; when the vehicle velocity is 80 km/h, the open degree of the sunroof is controlled to be 10%. Moreover, in the case where the sunroof is equipped with a tilt-up mechanism, when emitting an air cannon projectile, the sunroof may be maintained in a tilt-up state.

Moreover, a time difference may take place until an airflow in the compartment 2 is eliminated/reduced or the predetermined airflow state after the start of control of the open degree of the air conditioner is established. When the emission timing of the air cannon projectile is set to the same time as the control timing of the air conditioner, an air cannon projectile will be emitted while an airflow continues to be generated in the compartment 2, and consequently this air cannon projectile may be diffused. Therefore, it is preferable that the timing in which the open degree of the sunroof is controlled is set earlier than the control timing of the gaseous constituent emitting units 10-12, i.e., the emission timing of the air cannon projectile.

According to this embodiment, since the open degree of the sunroof is so controlled that an external airflow invading at the time of emission of the air cannon projectile is eliminated or reduced, the gaseous constituent can be supplied to the occupants 3, 4 when needed.

### Ninth Embodiment

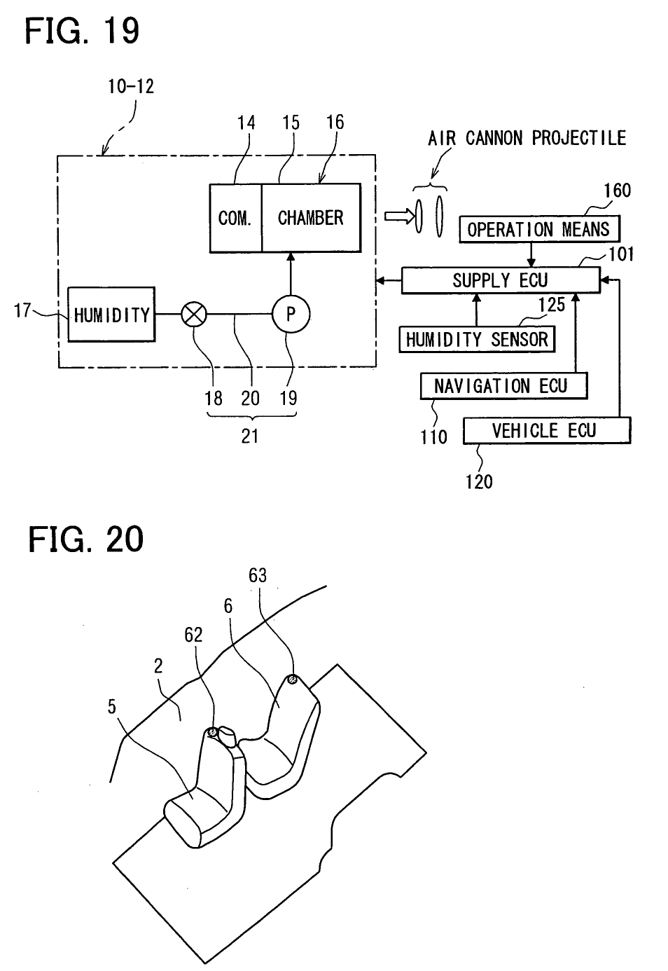

A ninth embodiment according to this invention will be described with reference to FIGS. 19-22. Description of the same parts as those of the above-mentioned first embodiment will be omitted, and only differences will be described. This embodiment differs from the first embodiment in that a humidity constituent generating unit 17 for generating a humidity constituent and a humidity sensor 125 for detecting the humidity of air in the compartment 2 are provided, and when a detection value of this humidity sensor 125 is not more than a predetermined value, an air cannon projectile is supplied to the compartment 2.

As shown in FIG. 19, the gaseous constituent supply chamber 15 of the gaseous constituent emitting units 10-12 is a space to which the conveyance pump 19 feeds the humidity constituent generated by the humidity generating means 17, letting it pass through the opened valve 18, via the connection tube 20, and where it is reserved. This space is connected with the conveyance means 21 so as to communicate with it. The humidity constituent conveyed by the conveyance means 21 is sent into the gaseous constituent supply chamber 15 as it is in a volatile state and made to fill this volume. When the above-mentioned humidity constituent is made to fill the gaseous constituent supply chamber 15 more than necessary, excessive humidity constituent will be emitted to the outside from the emission port 27. However, after the excessive humidity constituent leaks, humidity constituent more than that quantity will not leak immediately and a suitable quantity of the humidity constituent will be retained in the gaseous constituent supply chamber 15.

When a voltage is applied to the air compression means 14 from in-vehicle battery or the like, a current flows in the coil 24 and exerts electromagnetic force that pushes out the compression member 22 to the gaseous constituent supply chamber 15 side. Moreover, the magnet 23 also exerts a magnetic force that passes through tubular inside of the coil 24, and accordingly the compression member 22 deforms largely to the gaseous constituent supply chamber 15 side. The compression member 22 will project to the gaseous constituent supply chamber 15 side, up to the position 25, with a central part of the compression member 22 being protruded largely, and the coil 24 will project to the gaseous constituent supply chamber 15 side, up to the position 26, together with the compression member 22. Since this deformation of the compression member 22 makes small the volume of the gaseous constituent supply chamber 15 instantaneously, the above-mentioned humidity constituent filling this volume will be compressed and emitted to the outside from the emission port 27 instantaneously. By giving rise to this series of operations in a very short time, air containing the humidity constituent emitted from the emission port 27 is formed into a mass of a fluid, for example, in the form of a vortex ring, a sphere, etc. and emitted into the compartment 2.

The humidity sensor 125 (FIG. 19) is a sensor for detecting the humidity of air in the compartment 2. The humidity sensors 125 are arranged in backrest upper parts 62, 63 of the front seat 5 and the rear seat 6, as shown in FIG. 20. By disposing the humidity sensor 125 in the backrest upper part 62, the humidity data detected by the humidity sensor 125 is considered as the humidity of air that the occupant 3 sitting in the front seat 5 senses, and it becomes possible to detect accurately the degree of dryness of mucous membranes of the nose and throat of the occupant 3. Similarly, by disposing the humidity sensor 125 in the backrest upper part 63, the humidity data detected by the humidity sensor 125 is considered as the humidity of air that the occupant 4 sitting in the rear seat 6 senses, and it becomes possible to detect accurately the degree of dryness of mucous membranes of the nose and throat of the occupant 4. The humidity data detected by the humidity sensors 125 is sent to the gaseous constituent supply ECU 101.

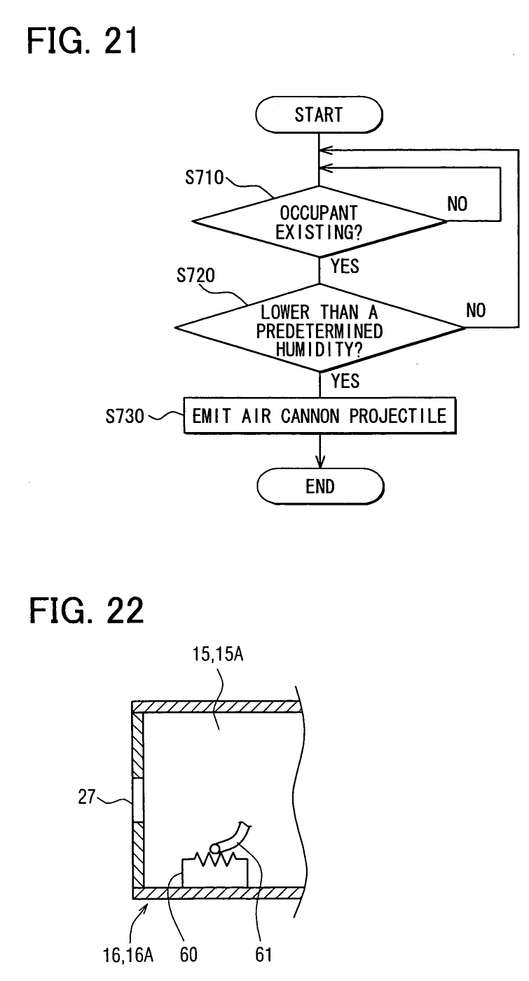

Next, a processing procedure whereby the gaseous constituent emitting units 10-12 are controlled based on the detection values of the humidity sensors 125 will be explained using FIG. 21. First, the gaseous constituent supply ECU 101 determines whether the occupant exists in the compartment 2 in Step S710. Regarding the existence of the occupant, the existence of the occupant shall be determined, for example, in the following cases: a case where an ignition switch is set on; a case where the infrared sensor detects heating from the occupant; a case where a weight sensor of a seat detects a predetermined or more weight; and a case where a predetermined or more value of CO₂ concentration is detected.

When it is determined that no occupant exists, the flow returns to Step S710 and repeats the same operation. When it is determined that the occupant exists, it is determined in Step S720 whether the detection value of the humidity sensor 125 is not more than a predetermined value. When the detection value is higher than the predetermined value, the ECU 101 determines that the humidity state in the compartment 2 is not in an unsuitable state, and the control process returns to Step S710, where the processing procedure to determine the existence of the occupant and a situation indicated by the detection value of the humidity sensor 125 is executed.

When it is determined that the detection value of the humidity sensor 125 is not less than a predetermined value, the control process moves to Step S730, where the gaseous constituent emitting units 10-12 is activated to emit an air cannon projectile toward the occupants or a certain space in the compartment 2. Then, the control process returns to the start and executes Step S710 and the following steps again. At this time, in the case where the plurality of humidity sensors 125 are arranged in the compartment 2, the gaseous constituent supply ECU 101 may perform both or either of a control that emits an air cannon projectile toward the occupant existing in a location nearest to the humidity sensor 125 having detected a predetermined or more value or the most nearest space and a control that activates the gaseous constituent emitting units 10-12 existing in a location nearest to that humidity sensor.

Since this processing enables the humidity constituent to be supplied in a coupled manner with an in-vehicle air state, an environment of the occupant existing in a location near the detection position of the humidity sensor 125 can be improved properly.

As shown in FIG. 22, the humidity constituent generating unit 17 may includes a water supply unit 61 and a heating unit 60 for evaporating the water supplied from the water supply unit 61, and may be installed in the gaseous constituent supply chamber 15, 15A. The water supply unit 61 is made up of tube, capable of providing water from the outside. The heating unit 60 is a heater for heating immediately water flowing out from the tube, and vaporizes the water to make air in the gaseous constituent supply chamber 15, 15A highly humid.

Thus, by constructing the humidity generating means 17 being integral with the enclosure 16, 16A, it becomes unnecessary to provide the enclosure having the humidity generating means 17 individually in it in contrast to a configuration where the enclosure 16 is connected with the humidity generating means 17 through the conveyance means 21, as shown in FIG. 19; therefore, the device can be miniaturized.

### Other Embodiments

This invention is not limited to the embodiments explained by the above-mentioned description and drawings. For example, the following embodiments are included within a technological scope of this invention. Further, the embodiments may be carried out after being modified in various manners within a scope that does not deviate from the gist.

In the above-mentioned embodiments, an air cannon projectile containing a perfume constituent as a gaseous constituent or an air cannon projectile containing the humidity constituent is emitted toward the occupants 3, 4. However, not limited to this, an air cannon projectile that contains both a perfume constituent as a gaseous constituent and the humidity constituent may be emitted toward the occupants 3, 4. Moreover, the gaseous constituent may be air alone.

Moreover, other embodiments may be carried out by combining any two or more of the above-mentioned first embodiment to the ninth embodiment arbitrarily.

What is claimed is:

1. A gaseous constituent supply device for supplying an air cannon projectile containing a predetermined gaseous constituent to an occupant in a compartment of a vehicle, comprising:
a gaseous constituent supply chamber for reserving the gaseous constituent;
an air compression means for compressing an inside of the gaseous constituent supply chamber to generate an air cannon projectile including the gaseous constituent and to emit the air cannon projectile to an individual occupant; and
a control unit that estimates an airflow state in the compartment and controls operation of the air compression means based on the estimated airflow state.

2. The gaseous constituent supply device according to claim 1, wherein:
the control unit permits operation of the air compression means when a set air volume of air-conditioning air being blown from an outlet port of an air conditioner of the vehicle is less than a predetermined amount; and
the control unit stops the operation of the air compression means when the set air volume is larger than the predetermined amount.

3. The gaseous constituent supply device according to claim 1, wherein:
the control unit permits operation of the air compression means when a blowing direction of air-conditioning air being blown from an outlet port of an air conditioner is set to be in a direction diverted from the occupant; and
the control unit stops the operation of the air compression means when the blowing direction is set to be in a direction toward the occupant.

4. The gaseous constituent supply device according to claim 1, wherein:
the control unit permits operation of the air compression means when a window glass of the vehicle is set in a close state; and
the control unit stops the operation of the air compression means when the window glass is set in an open state.

5. The gaseous constituent supply device according to claim 1, wherein,
the control unit permits operation of the air compression means when a sunroof of the vehicle is set in a close state; and
the control unit stops the operation of the air compression means when the sunroof of the vehicle is set in an open state.

6. The gaseous constituent supply device according to claim 1, further comprising
an airflow sensor for detecting an airflow, wherein:
the control unit permits operation of the air compression means when the control unit determines a non-airflow state in the compartment based on the detected value of the airflow sensor; and
the control unit stops the operation of the air compression means when the control unit estimates an airflow state in the compartment based on the detected value of the airflow sensor.

7. The gaseous constituent supply device according to claim 6, wherein:
the airflow sensor is disposed to detect a flow velocity of the airflow;
the control unit permits the operation of the air compression means when the control unit estimates that the flow velocity of the airflow is not more than a predetermined velocity; and
the control unit stops the operation of the air compression means when the control unit estimates that the flow velocity is more than the predetermined velocity.

8. The gaseous constituent supply device according to claim 1, further comprising
a state detecting sensor for detecting a seating state of the occupant,
wherein the control unit controls operation of the air compression means depending on a change in the seating state.

9. The gaseous constituent supply device according to claim 1, wherein:
the control unit estimates an airflow state, based on an operating state of an in-vehicle apparatus in a timing in which the air cannon projectile is emitted; and
the control unit controls an operation of the air compression means such that the air cannon projectile reaches the occupant even in an airflow generation state in which an airflow is generated in the compartment when the control unit estimates the airflow generation state.

10. The gaseous constituent supply device according to claim 9, wherein:
the in-vehicle apparatus is an air conditioner for air-conditioning the compartment by blowing air-conditioning air to the compartment; and
the control unit estimates the airflow state based on at least one of a blowing direction and an air volume of the air-conditioning air.

11. The gaseous constituent supply device according to claim 9, wherein
the in-vehicle apparatus is a window glass of the vehicle, and the control unit estimates the airflow state based on an open degree of the window glass.

12. The gaseous constituent supply device according to claim 9, wherein
the in-vehicle apparatus is a sunroof of the vehicle, and the control unit estimates the airflow state based on the open degree of the sunroof.

13. The gaseous constituent supply device according to claim 1, wherein:
the control unit estimates an airflow state in the compartment based on an operating state of an in-vehicle apparatus in a timing of emitting the air cannon projectile;
when it is estimated to be an airflow generation state in which an airflow is generated in the compartment, the control unit reduces the airflow generation state; and
the control unit controls an operating state of the in-vehicle apparatus such that the air cannon projectile reaches the occupant even in the airflow generation state.

14. The gaseous constituent supply device according to claim 13, wherein:
the in-vehicle apparatus is an air conditioner for air-conditioning the compartment by blowing air-conditioning air to the compartment;
the control unit presumes the airflow state in the compartment based on at least one of a blowing direction and an air volume of the air-conditioning air; and
when the control unit estimates that the airflow state is an airflow generation state, the control unit controls at least one of the blowing direction and the air volume of the air-conditioning air.

15. The gaseous constituent supply device according to claim 14, wherein:
the control unit controls the air conditioner such that the blowing direction of the air-conditioning air approaches the same direction as an emission direction of the air cannon projectile and a flow velocity of the air-conditioning air approaches the same as a flow velocity of the air cannon projectile.

16. The gaseous constituent supply device according to claim 13, wherein:
the in-vehicle apparatus is a window glass of the vehicle; and
the control unit reduces an open degree of the window glass.

17. The gaseous constituent supply device according to claim 13, wherein:
the in-vehicle apparatus is a sunroof of the vehicle; and
the control unit reduces an open degree of the sunroof.

18. The gaseous constituent supply device according to claim 13, wherein
the control unit controls the in-vehicle apparatus before starting of an operation of the air compression means.

19. The gaseous constituent supply device according to claim 1, wherein the gaseous constituents include a plurality of perfume constituents, further comprising
perfume constituent supply means for supplying selectively the plurality of perfume constituents as the air cannon projectile to the gaseous constituent supply chamber.

20. The gaseous constituent supply device according to claim 19, wherein
the control unit determines a perfume constituent selected from among the plurality of perfume constituents based on a signal of a navigation ECU of the vehicle or a signal of a vehicular integrated ECU.

21. The gaseous constituent supply device according to claim 19, wherein
the control unit determines a perfume constituent selected from among the plurality of perfume constituents based on detection data of a temperature sensor for detecting a body temperature of the occupant.

22. The gaseous constituent supply device according to claim 19, wherein
the control unit determines a perfume constituent selected from among the plurality of perfume constituents based on detection data of a biomedical sensor for detecting a biomedical signal of the occupant.

23. The gaseous constituent supply device according to claim 1, further comprising
an operation selection switch capable of selecting an operation or operation stop of the air compression means,
wherein the control unit operates based on an item being set up by the operation selection switch.

24. The gaseous constituent supply device according to claim 19, further comprising
a perfume constituent selection switch capable of selecting at least one perfume constituent from among the plurality of perfume constituents,
wherein the perfume constituent supply means operates based on an item being set up by the perfume constituent selection switch.

25. The gaseous constituent supply device according to claim 1, wherein:
the gaseous constituent supply chamber and the air compression means are constructed in an enclosure; and
the air compression means includes a compression member for changing a volume of the gaseous constituent supply chamber by magnetic force and electromagnetic force, a magnet provided in the compression member, and a coil installed facing the compression member.

26. The gaseous constituent supply device according to claim 1, wherein the gaseous constituents include a humidity constituent, further comprising:
a humidity constituent generating unit for generating a humidity constituent; and
a humidity sensor for detecting a humidity of air in the compartment,
wherein the control unit controls the humidity constituent generating unit to supply the air cannon projectile including the humidity constituent into the compartment when a detection value of the humidity sensor is lower than a predetermined value.

27. A gaseous constituent supply device for supplying an air cannon projectile containing a predetermined gaseous constituent to an occupant in a compartment of a vehicle, comprising:
a humidity constituent generating unit for generating a humidity constituent to be contained in the air cannon projectile;
a gaseous constituent chamber for reserving the gaseous constituent;
air compression means for compressing an inside of the gaseous constituent chamber to generate an air cannon projectile including the humidity constituent;
a humidity sensor for detecting a humidity of air in the compartment; and
a control unit which controls the humidity constituent generating unit and the compression means so that an air cannon projectile including a predetermined amount of the humidity constituent is emitted to the occupant when the humidity of air detected by the humidity sensor is lower than a predetermined value.

28. The gaseous constituent supply device according to claim 27, wherein:
the humidity constituent generating unit includes a water supply unit for supplying water and a heating unit for evaporating the water supplied from the water supply unit; and
the humidity constituent generating unit is disposed in the gaseous constituent chamber.

29. A gaseous constituent supply device for supplying an air cannon projectile containing a predetermined gaseous constituent to an occupant in a compartment of a vehicle, comprising:
an enclosure having therein a gaseous constituent chamber for reserving the gaseous constituent, and air compression means for compressing an inside of the gaseous constituent chamber to generate an air cannon projectile including the predetermined gaseous constituent, wherein:
the air compression means includes a compression member for changing a volume of the gaseous constituent chamber by magnetic force and electromagnetic force, a magnet provided in the compression member, and a coil installed facing the compression member.

30. The gaseous constituent supply device according to claim 29, wherein the compression member is a plate member having an elasticity.

31. The gaseous constituent supply device according to claim 29, wherein the electromagnetic force of the compression means is generated when a square-wave voltage is applied to the compression means.

32. The gaseous constituent supply device according to claim 31, wherein the compression means has a booster circuit through which the square-wave voltage is applied.
